(19) United States
(12) Patent Application Publication
(10) Pub. No.: US 2024/0374580 A1
(43) Pub. Date: Nov. 14, 2024

(54) SMALL MOLECULE INHIBITORS OF AUTOPHAGY AND HISTONE DEACTYLASES AND USES THEREOF

(71) Applicant: ARIZONA BOARD OF REGENTS ON BEHALF OF THE UNIVERSITY OF ARIZONA, Tucson, AZ (US)

(72) Inventors: Jennifer CAREW, Tucson, AZ (US); Steffan NAWROCKI, Tucson, AZ (US); Wei WANG, Tucson, AZ (US)

(21) Appl. No.: 18/564,323

(22) PCT Filed: May 12, 2022
(86) PCT No.: PCT/US2022/028970

§ 371 Date: Nov. 27, 2023

Related U.S. Application Data
(60) Provisional application No. 63/194,472, filed on May 28, 2021.

(51) Int. Cl. A61K 31/4709; A61K 31/4706; A61K 31/675; A61K 45/06; A61P 11/00; C07D 409/12

(52) U.S. Cl. CPC A61K 31/4709; A61K 31/4706; A61K 31/675; A61K 45/06; A61P 11/00; C07D 409/12

(57) ABSTRACT

This invention is in the field of medicinal chemistry. In particular, the invention relates to a new class of small-molecules having a tetrahydroacridine (or similar) structure, an acridine (or similar) structure, a benzonaphthyridine (or similar) structure, or a tetrahydrobenzonaphthyridine (or similar) structure which function as autophagy inhibitors and/or histone deactylase inhibitors, and their use as therapeutics for the treatment of conditions characterized with aberrant autophagy activity and/or aberrant HDAC activity (e.g., cancer, pulmonary hypertension, diabetes, neurodegenerative disorders, aging, heart disease, rheumatoid arthritis, infectious discuses, conditions and symptoms caused by a viral infection (e.g., COVID-19)).

### CROSS REFERENCE TO RELATED APPLICATION

This application claims priority to and the benefit of U.S. Provisional Application No. 63/194,472, filed May 28, 2021, which is hereby incorporated by reference in its entirety.

### FIELD OF THE INVENTION

This invention is in the field of medicinal chemistry. In particular, the invention relates to a new class of small-molecules having a tetrahydroacridine (or similar) structure, an acridine (or similar) structure, a benzonaphthyridine (or similar) structure, or a tetrahydrobenzonaphthyridine (or similar) structure which function as autophagy inhibitors and/or histone deactylase inhibitors, and their use as therapeutics for the treatment of conditions characterized with aberrant autophagy activity and/or aberrant HDAC activity (e.g., cancer, pulmonary hypertension, diabetes, neurodegenerative disorders, aging, heart disease, rheumatoid arthritis, infectious diseases, conditions and symptoms caused by a viral infection (e.g., COVID-19)).

### INTRODUCTION

Autophagy consists of the sequestration of organelles and proteins in autophagic vesicles and degradation of this cargo through lysosomal fusion (see, Lum J J, et al., Nat Rev Mol Cell Biol. 2005: 6 (6): 439-48). Autophagy allows tumor cells to survive metabolic and therapeutic stresses (see, Amaravadi R K, and Thompson C B. Clin Cancer Res. 2007: 13 (24): 7271-9; Amaravadi R K, et al., J Clin Invest. 2007: 117 (2): 326-36: Degenhardt K, et al., Cancer Cell. 2006: 10 (1): 51-64; Amaravadi R K., et al., J Clin Invest. 2008; Carew J S, et al., Blood. 2007). Multiple publications indicate therapy-induced autophagy is a key resistance mechanism to many anti-cancer agents. Chloroquine (CQ) derivatives block autophagy by inhibiting the lysosome (see, Amaravadi R K, et al., J Clin Invest. 2007: 117 (2): 326-36; Carew J S, et al., Blood. 2007; Degtyarev M, et al., J Cell Biol. 2008; 183 (1): 101-16). Based on these findings, clinical trials combining cancer therapies with hydroxy chloroquine (HCQ) have been launched. Results indicate these combinations have activity (see, Amaravadi R K, et al., Clin Cancer Res. 2011: 17 (4): 654-66; Rebecca V W, et al., Pigment Cell Melanoma Res. 2014: 27 (3): 465-78: Mahalingam D, et al., Autophagy. 2014: 10 (8); Rangwala R, et al., Autophagy. 2014: 10 (8): Rangwala R, et al., Autophagy. 2014; 10 (8); Rosenfeld M R, et al., Autophagy. 2014; 10 (8)), but it is still unclear if this activity is consistently due to the addition of HCQ. High micromolar concentrations of HCQ are required to inhibit autophagy. While there is some pharmacodynamic evidence of autophagy inhibition with HCQ in cancer patients, it is inconsistent because adequate concentrations are not achieved in all patients.

As such, there is an unmet need to develop more potent inhibitors of autophagy.

The present invention addresses this need.

### SUMMARY OF THE INVENTION

Experiments conducted during the course of developing embodiments for the present invention designed, synthesized and biologically evaluated compounds having a tetrahydroacridine (or similar) structure, an acridine (or similar) structure, a benzonaphthyridine (or similar) structure, or a tetrahydrobenzonaphthyridine (or similar) structure as autophagy inhibitors and/or histone deactylase (HDAC) inhbitors, and their potential for use as therapeutics against conditions characterized with aberrant autophagy activity and/or aberrant HDAC activity (e.g., cancer, pulmonary hypertension, diabetes, neurodegenerative disorders, aging, heart disease, rheumatoid arthritis, infectious diseases, etc.).

Certain compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, compounds encompassed within Formula I are provided:

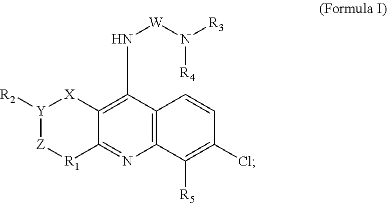

including pharmaceutically acceptable salts, solvates, and/or prodrugs thereof.

Formula I is not limited to a particular chemical moiety for W, X, Y, Z, R₁, R₂, R₃, R₄, and R₅. In some embodiments, the particular chemical moiety for W, X, Y, Z, R₁, R₂, R₃, R₄, and R₅ independently include any chemical moiety that permits the resulting compound to inhibit cellular autophagy. In some embodiments, the particular chemical moiety for W, X, Y, Z, R₁, R₂, R₃, R₄, and R₅ independently include any chemical moiety that permits the resulting compound to inhibit cellular HDAC activity. In some embodiments, the particular chemical moiety for W, X, Y, Z, R₁, R₂, R₃, R₄, and R₅ independently include any chemical moiety that permits the resulting compound to inhibit cellular autophagy and cellular HDAC activity. In some embodiments, the particular chemical moiety for W, X, Y, Z, R₁, R₂, R₃, R₄, and R₅ independently include any chemical moiety that permits the resulting compound to act as an effective therapeutic for treating conditions characterized with aberrant autophagy activity and/or aberrant HDAC activity (e.g., cancer, pulmonary hypertension, diabetes, neurodegenerative disorders, aging, heart disease, rheumatoid arthritis, infectious diseases, conditions and symptoms caused by a viral infection (e.g., COVID-19)).

Such embodiments are not limited to particular definition for W. In some embodiments, W is selected from

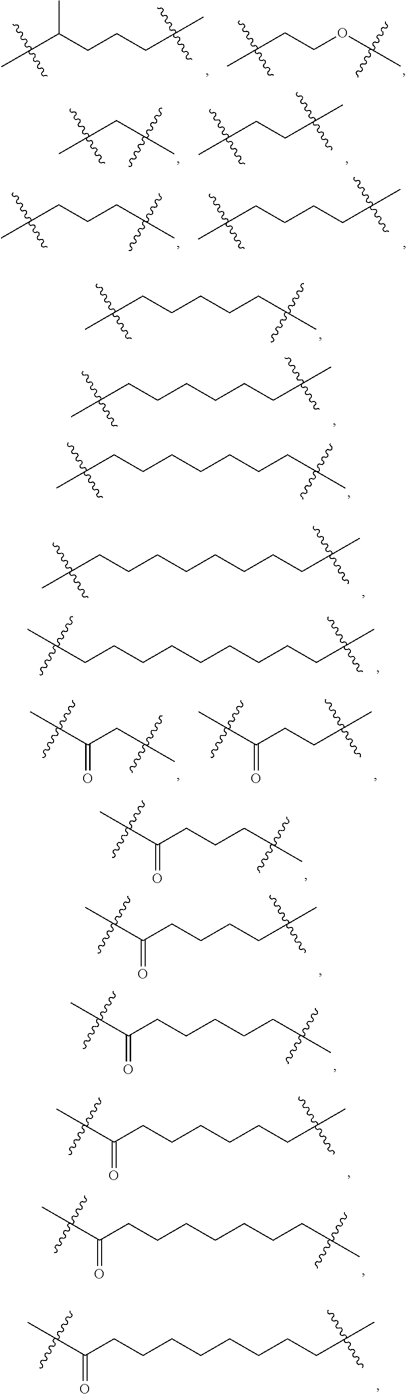

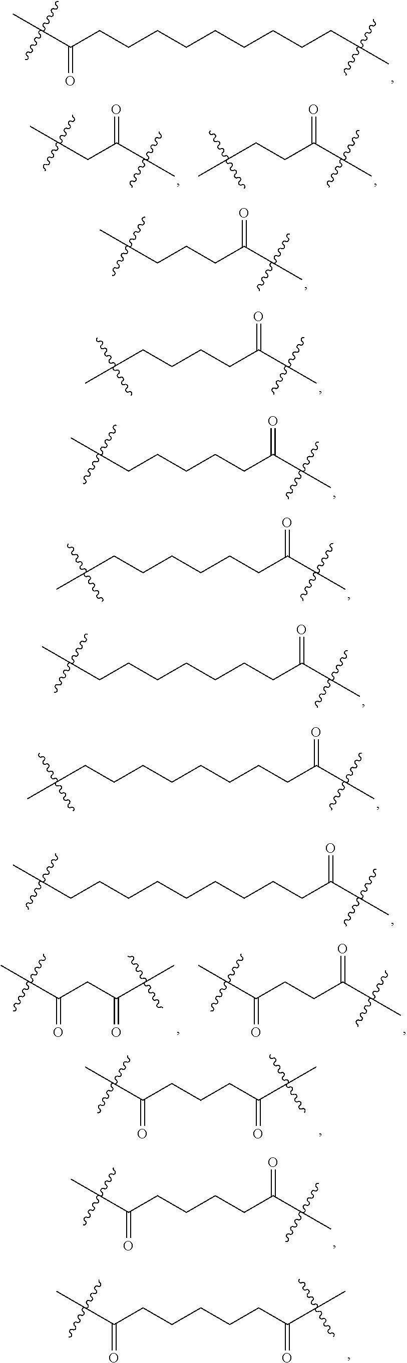

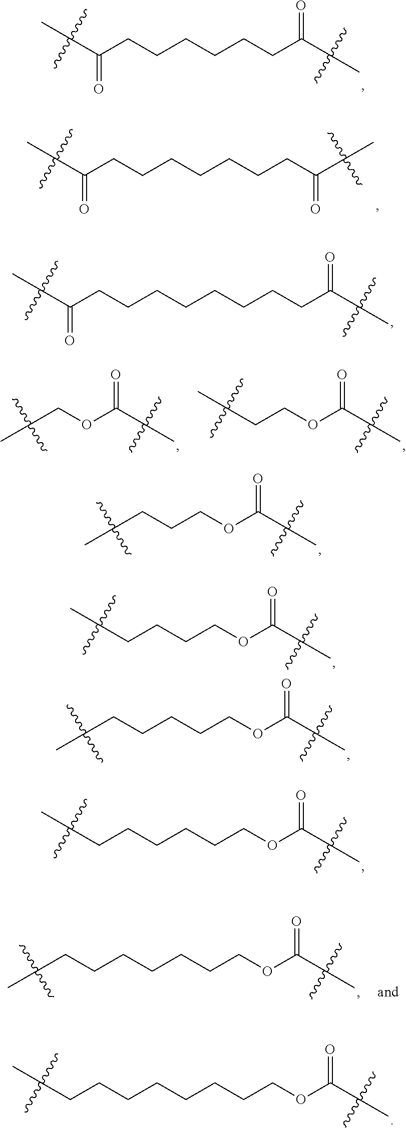

Such embodiments are not limited to a particular definition for

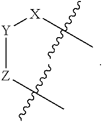

In some embodiments, each of X, Y, and Z are CH or N such that the resulting compound is represented by Formula II:

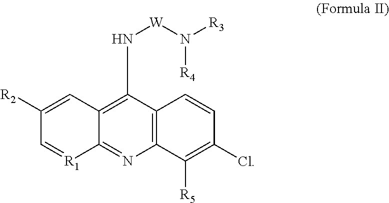

In some embodiments, each of X and Z are CH₂, N, and Y is CH such that the resulting compound is represented by Formula III:

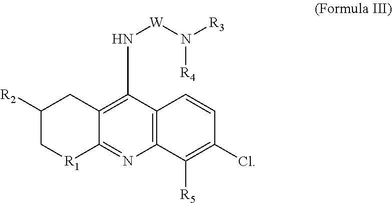

Such embodiments are not limited to a particular definition for R₁. In some embodiments, R₁ is CH₂, N-alkyl, aryl or NH or NCO—.

Such embodiments are not limited to a particular definition for R2. In some embodiments, R₂ is methoxy, halogens, NH₂, alkyl, aryl, or hydrogen.

Such embodiments are not limited to a particular definition for R₃. In some embodiments, R₃ is selected from hydrogen, methyl, ethyl, Ph,

pyridine

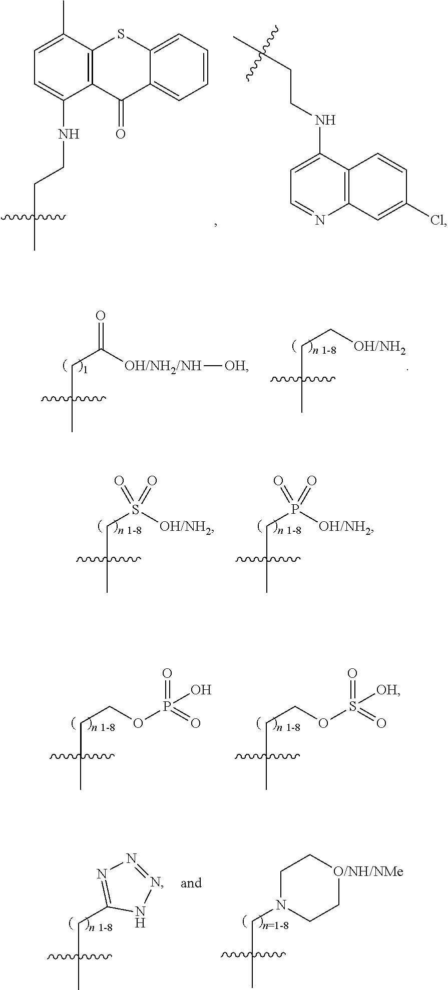

Such embodiments are not limited to particular definition for R₄.

In some embodiments. R₄ is selected from hydrogen, methyl, ethyl, hydroxyl.

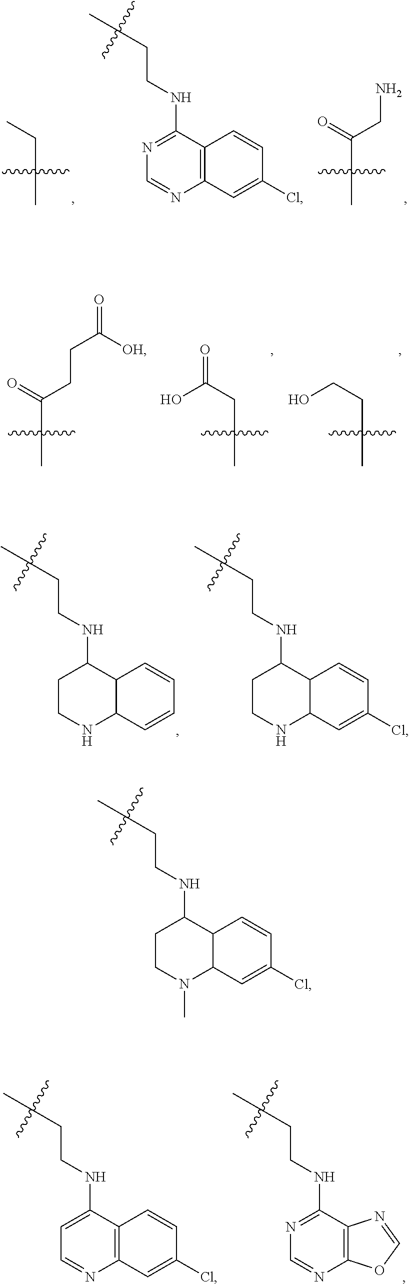

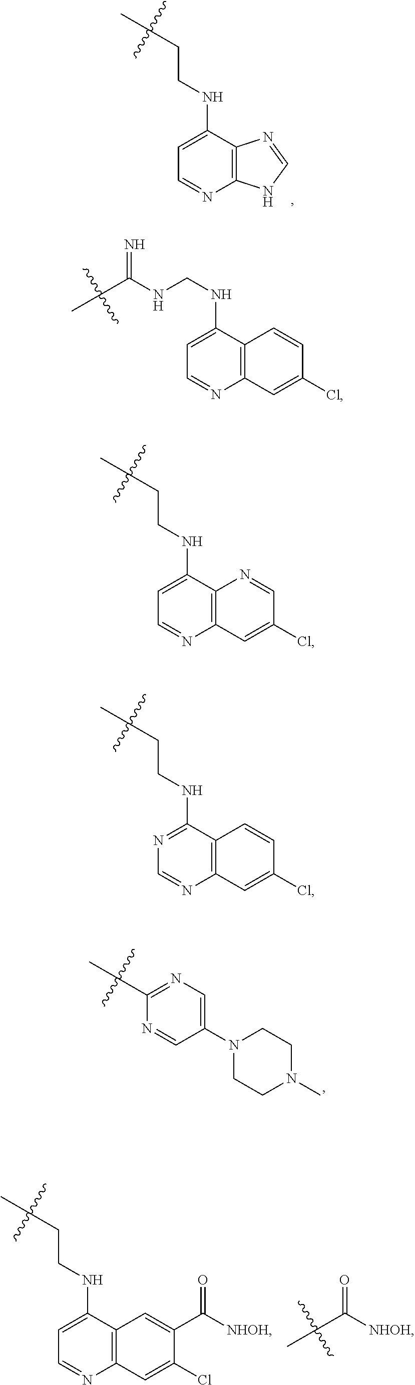

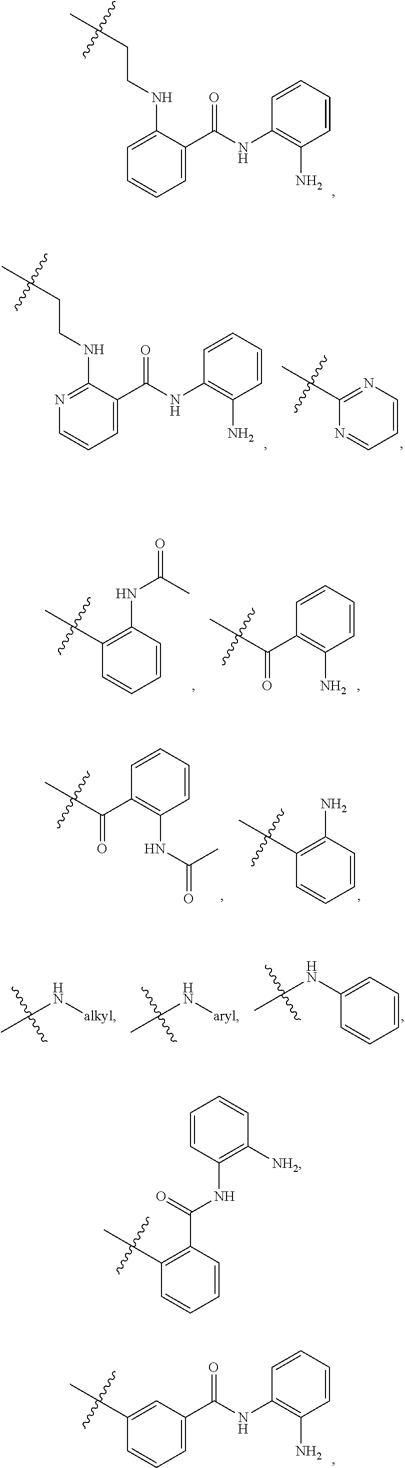

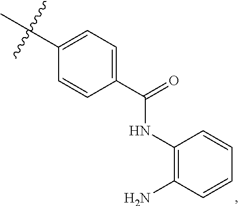 [SMILES: *c1ccc(C(=O)Nc2ccccc2N)cc1]

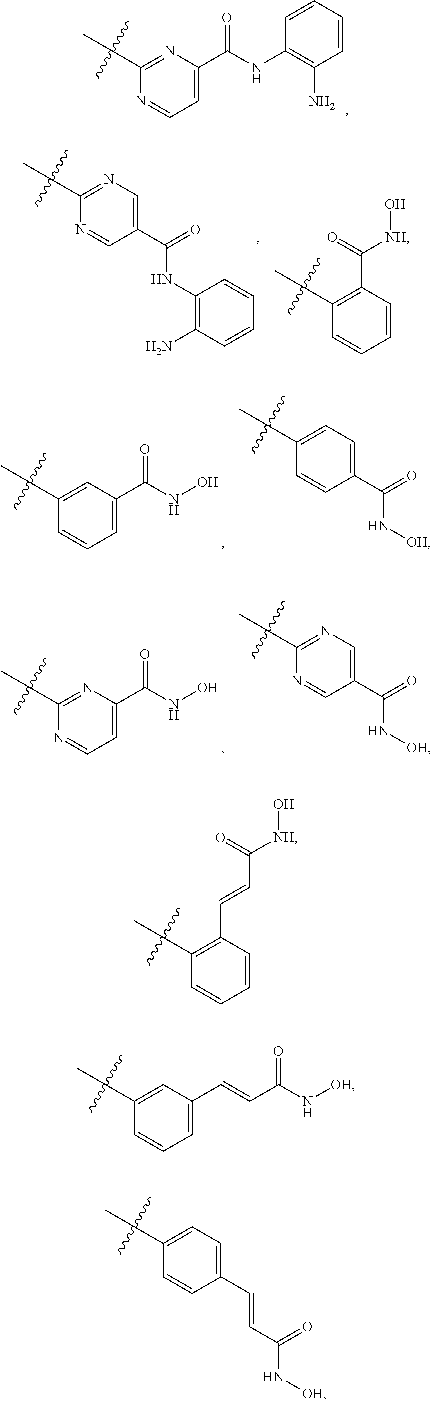

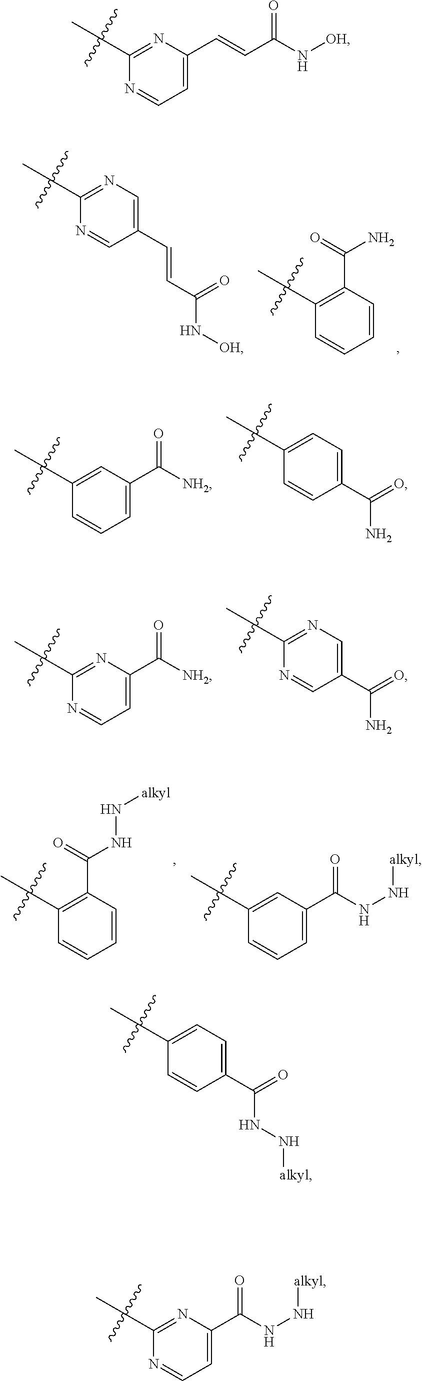

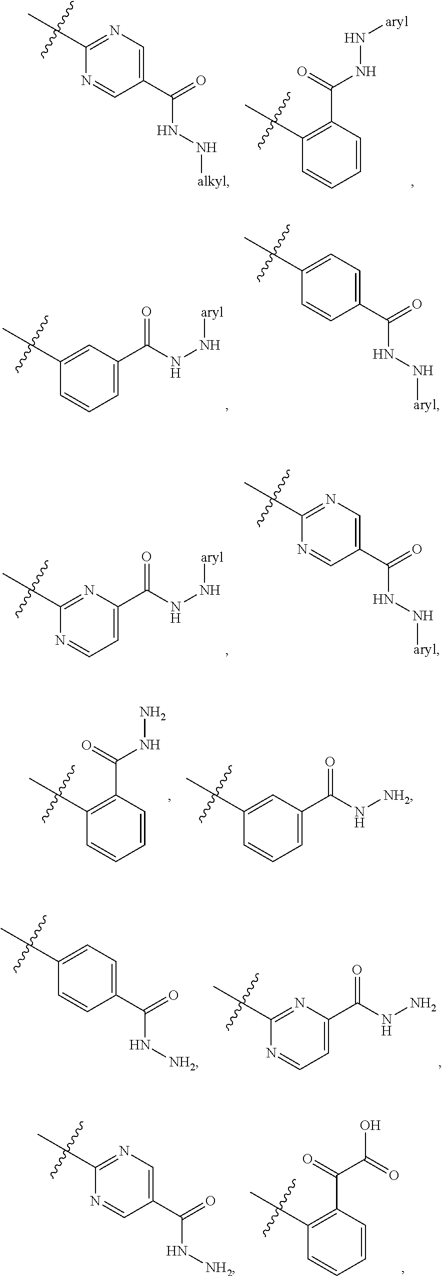

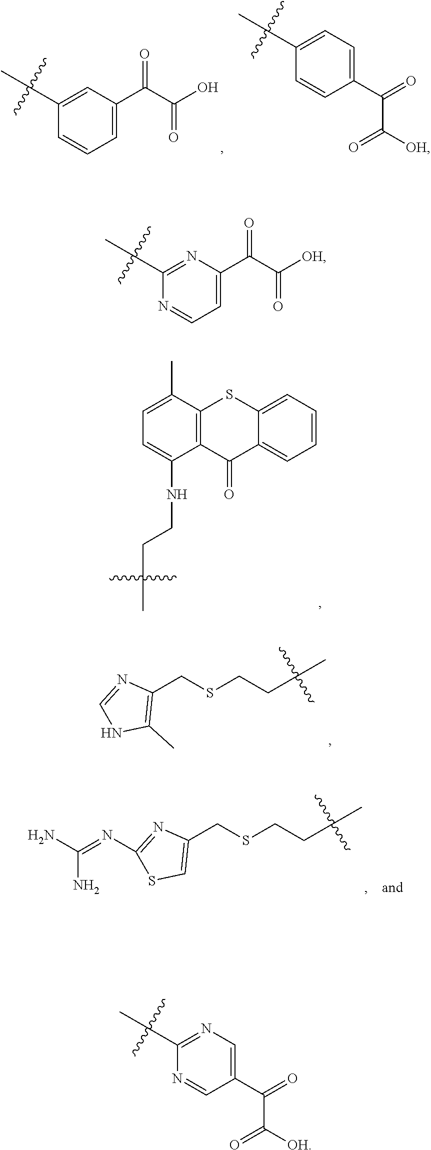

Such embodiments are not limited to particular definition for R₅. In some embodiments, R₅ is selected from hydrogen, methyl, and deuterated methyl (e.g., CD3).

The invention further provides processes for preparing any of the compounds of the present invention.

As noted, the compounds of the invention are useful for the treatment, amelioration, or prevention of conditions characterized with aberrant autophagy activity and/or aberrant HDAC activity (e.g., cancer, pulmonary hypertension, diabetes, neurodegenerative disorders, aging, heart disease, rheumatoid arthritis, infectious diseases, conditions and symptoms caused by a viral infection (e.g., COVID-19)).

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., agents useful in treating conditions that respond favorably to autophagy inhibition and/or HDAC inhibition (e.g., cancer, pulmonary hypertension, diabetes, neurodegenerative disorders, aging, heart disease, rheumatoid arthritis, infectious diseases, conditions and symptoms caused by a viral infection (e.g., COVID-19)).

Severe acute respiratory syndrome coronavirus 2 (SARS-COV-2) is an enveloped, positive-sense, single-stranded RNA beta-coronavirus that emerged in Wuhan in November 2019 and rapidly developed into a global pandemic. The associated disease, COVID-19, has an array of symptoms, ranging from flu-like illness and gastrointestinal distress (Xiao et al. 2020; Lin et al. 2020) to acute respiratory distress syndrome, heart arrhythmias, strokes, and death (Avula et al. 2020; Kochi et al. 2020). Drug repurposing has played an important role in the search for COVID-19 therapies. Recently, the FDA issued emergency approval of remdesivir (GS-5734), a monophosphoramidate prodrug of a nucleoside inhibitor developed for Ebola virus treatment (Mulangu et al. 2019), and hydroxychloroquine, an aminoquinoline derivative first developed in the 1940s for the treatment of malaria, for severely ill patients with COVID-19. However, there are no established prophylactic strategies or direct antiviral treatments available to limit SARS-COV-2 infections and to prevent/cure the associated disease COVID-19.

Prophylactic strategies and/or direct antiviral treatments for preventing, treating and ameliorating the symptoms of SARS-COV-2/COVID-19 are desperately needed.

In certain embodiments, the present invention provides methods for administering a pharmaceutical composition comprising one or more of the compounds of the present invention to a subject (e.g., a human subject) (e.g., a human subject suffering from or at risk of suffering from a condition related to SARS-COV-2 infection (e.g., COVID-19)) for purposes of treating, preventing and/or ameliorating the symptoms of a viral infection (e.g., SARS-COV-2 infection (e.g., COVID-19)).

In such embodiments, the methods are not limited treating, preventing and/or ameliorating the symptoms of a particular type or kind of viral infection. In some embodiments, the viral infection is a SARS-COV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virals having MPro protease activity and/or expression.

In certain embodiments, the present invention provides methods for treating, ameliorating and/or preventing a condition related to viral infection in a subject, comprising administering to the subject a pharmaceutical composition comprising one or more of the compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-COV-2 infection (e.g., COVID-19). In some embodiments, the viral infection is a SARS-COV-2 viral infection.

In certain embodiments, the present invention provides methods for treating, ameliorating and/or preventing symptoms related to viral infection in a subject, comprising administering to the subject a pharmaceutical composition comprising one or more of the compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-COV-2 infection (e.g., COVID-19). In some embodiments, the subject is a human subject suffering from a SARS-COV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising one or more of the compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-COV-2 infection (e.g., COVID-19). In some embodiments, the subject is a human subject suffering from a SARS-COV-2 viral infection.

In certain embodiments, the present invention provides methods for treating, ameliorating and/or preventing pneumonia in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-COV-2 infection (e.g., COVID-19). In some embodiments, the subject is a human subject suffering from a SARS-COV-2 viral infection.

In some embodiments involving the treatment of acute respiratory distress syndrome and/or pneumonia, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include H1 antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisterization by nebulization, anti-tussives, respiratory stimulants, surgery and alpha 1 antitrypsin.

In certain embodiments, the present invention provides kits comprising (1) a pharmaceutical composition comprising one or more compounds of the present invention, (2) a container, pack, or dispenser, and (3) instructions for administration. In some embodiments, the kit further comprises remdesivir or hydroxychloroquine.

### BRIEF DESCRIPTION OF THE DRAWINGS

FIG. 1. Autophagy promotes PAH pathogenesis by generating alternative sources of metabolic fuel. Cells use autophagy as a mechanism of metabolic reprogramming to lysosomally catabolize proteins and organelles to generate alternative sources of energy. Autophagy provides fuel for disease progression and cell survival to overcome stress caused by hypoxia and other factors.

FIG. 2A-E. Vasorelaxant effect of chloroquine on rings of rat PA. (A) Isometric tension in an isolated PA ring constricted with 1 μM phenylephrine (PE) before and during application of chloroquine (CHQ or CQ). (B) Dose-response curve of CHQ induced vasorelaxation in rings of rat PA. pre-constricted with 1 μM PE. n=12, mean±SEM. (C) Isometric tension in a PA ring consecutively contracted with 1 μM PE before, during and after application of 200 μM CHQ. (D) Isometric tension in a PA ring constricted with 40 mM (40K) or 80 mM (80K) K+-containing solution before, during and after application of 200 μM CHQ. (E) Summarized data (mean±SE) showing decreased active tension induced by 200 μM CHQ in rings of rat PA pre-contracted with 40K or 80K. n=6 per group. * P<0.05.

FIG. 3A-E. Vasorelaxant effect of CHQ on isolated rat PA is endothelium-independent. (A and B) Effect of 200 μM CHQ on isometric tension in endothelium-intact (A) or endothelium-denuded (B) PA rings constricted with 1 μM PE. (C) Summarized data showing that CHQ (200 μM) induced decreases in active tension in endothelium-intact or endothelium-denuded rat PA rings pre-constricted with 1 μM PE. N=6, mean±SEM. (D) Effect of 200 μM CHQ on PE-induced active tension in PA rings pretreated (for 30 min) with the PGI2 synthesis inhibitor, indomethacin (INDO: 10 μM) or the endothelial NOS inhibitor, 1-NAME (100 μM). (E) Summarized data showing CHQ (200 μM)-induced decrease in PE-induced active tension in PA rings pretreated with vehicle (n=6), 1-NAME (n=8) or INDO (n=6), mean±SEM.

FIG. 4A-G. CHQ inhibits store-operated, ROCE and reduces whole-cell Ca2+ currents in human PASMC. (A) Changes in [Ca2+]cyt in control PASMC and CHQ (200 μM)-treated PASMC before, during and after application of 10 μM CPA (an inhibitor of SERCA) in the absence or presence of extracellular Ca2+ (1.8 mM). (B) Summarized data showing the amplitude of increase in [Ca2+]cyt induced by application of 10 μM CPA in the absence (first peak) or presence (second peak) of 1.8 mM extracellular Ca2+ (n=44-54 PASMC). Mean±SEM, *P<0.05 (C) Changes in [Ca2+]cyt before and during application of 100 μM ATP in control PASMC and CHQ (200 μM)-treated human PASMC. (D) Summarized data showing [Ca2+]cyt before (basal) and during (ATP) application of ATP in control and CHQ-treated PASMC. Means±SEM, *P<0.05. (E) Summarized data showing ATP-induced increases in [Ca2+]cyt in control and CHQ-treated PASMC. means±SEM *P<0.05. (F) Representative record (left panel) of inward currents, elicited by a depolarizing test pulse of +10 mV from a holding potential of −70 mV, in PASMC before (Cont), during (CHQ) and after (wash) extracellular application of CHQ. Right panel: summarized data showing the amplitudes of peak inward currents at +10 mV in PASMC before (Cont), during (CHQ) and after (wash) extracellular application of CHQ (n=6 PASMC). Means±SEM, *P<0.05. (G) The current-voltage (I-V) relationship curves constructed from current recordings in PASMC before (Cont), during (CHQ) and after (wash) application of CHQ.

FIG. 5A-C. CHQ attenuates pulmonary vasoconstriction induced by high K+ or hypoxia. (A) Left, PAP in isolated perfused and ventilated lungs before, during and after repeated pulmonary arterial perfusion of 40 mM K+ (40K) solution. CHQ (200 μM) was applied to the lung via the intrapulmonary arterial catheter when a 40K-induced increase in PAP reached its maximal plateau phase. Right, summarized data showing the 40K-induced increases in PAP in isolated perfused and ventilated lungs before (control), during (CHQ) and after (washout) perfusion of the pulmonary circulation with CHQ (200 μM). n=6, mean±SEM), *P<0.05. (B) Left, 40K-induced changes of PAP in isolated perfused and ventilated lungs before, during and after intrapulmonary application of CHQ (200 μM). Right, summarized data showing the repeated 40K-induced increases in PAP before and during application of CHQ. n=5), mean±SEM, *P<0.05. (C) Left, alveolar hypoxia (Hyp)-induced changes of PAP in isolated perfused and ventilated lungs before, during and after intrapulmonary application of CHQ (200 μM). Right, summarized data showing the repeated Hyp-induced increases in PAP before and during application of CHQ. n=6, mean±SEM, *P<0.05.

FIG. 6A-B. CHQ-induced pulmonary vasodilation is not inhibited by blockade of K+ channels. (A) Isometric tension before and during application of CHQ (200 μM) in PE (1 μM)-contracted PA rings that were pretreated (for 30 min) with vehicle, 5 μM paxilline (a BK channel blocker), 10 mM TEA (a non-selective K+ channel blocker that blocks voltage-gated K+ channels and BK channels) or 100 nM ChTX (a BK and IK channel blocker). (B) Summarized data showing CHQ-induced decrease in active tension in PA rings treated with vehicle (n=6), 5 μM paxilline (n=5), 10 mM TEA (n=5) or 100 nM ChTX (n=5). Mean+ SEM.

FIG. 7A-E. CHQ inhibits autophagy and decreases proliferation of human PASMC. Representative Western blot (A) and (B) summarized quantification (n=5, mean±SEM) of P62 and LC3B in control and 20 μM CHQ-treated PASMC (24 h). * P<0.05. (C) LC3 immunofluorescence of control and 20 μM CHQ (24 h) treated PASMC. (D) Quantification of the % LC3 positive PASMC following 24 h 20 μM CHQ treatment, mean±SEM. (E) Dose-dependent effects of 48 h CHQ treatment on PASMC. Cell viability was determined by MTT assay. n=6, mean±SEM. * P<0.05.

FIG. 8A-F. CHQ inhibits the development of HPH in rats. (A) Right ventricular pressure (RVP) in rats exposed to normoxia alone (Nor) or with CHQ (50 mg/kg/day) and rats exposed to hypoxia (Hyp: 10% 02 for 21 days) or with CHQ (Hyp+CHQ) treatment. (B) Summarized data, as means #SEM, showing the peak value of RVSP in Nor, Nor+ CHQ, Hyp and Hyp+CHQ rats (n=6 per group). * P<0.05, significantly different from Nor and Nor+CHQ groups: #P<0.05, significantly different from Hyp group. (C) Summarized data, as means±SEM, showing the RV hypertrophy index, determined by the Fulton index [RV/(LV+S)](left panel) and the ratio of RV/BW (right panel) in Nor, Nor+CHQ, Hyp and Hyp+CHQ rats (n=6 per group). (D) Summarized data, as means±SEM, showing haematocrit (HCT) in Nor, Nor+CHQ, Hyp and Hyp+CHQ rats (n=6 in each group). *P<0.05, significantly different from Nor and Nor+CHQ groups: #P<0.05, significantly different from Hyp group. (E) Representative H&E images of small PAs in Nor, Nor+CHQ, Hyp and Hyp+CHQ rats. (F) Summarized data, as means±SEM, showing the wall area/vessel area, thickness and area of small PA in Nor, Nor+CHQ, Hyp and Hyp+CHQ rats. * P<0.05, significantly different from Nor and Nor+CHQ groups: #P<0.05, significantly different from Hyp group.

FIG. 9A-C. CHQ inhibits progression of established PH in the SuHx rat model. (A) Representative right ventricular pressure (RVP). Rats were given an s.c. injection of 20 mg/kg SU5416 or vehicle, followed by hypoxia (SuHx) for 3 weeks (10% 02) and normoxia for an additional 2 weeks to elicit severe PH. After 3 weeks of hypoxia, the SuHx rats were divided into three groups: hypoxia with no intervention (SuHx3w), vehicle treatment (SuHx3w+Nor2w) and treatment with CHQ (50 mg/kg/day, SuHx3w+CHQ2w). The normoxic control group was exposed to room air only (21% 02) for a total of 5 weeks (Nor). n=6 for each group. (B) Summarized data, as means±SEM, showing the peak value of RVSP in Nor, SuHx3w, SuHx3w+Nor2w and SuHx3w+CHQ2w groups. * P<0.05, significantly different from Nor: #P<0.05, significantly different from SuHx3w+Nor2w. (C) Summarized data, as means±SEM, showing the RV hypertrophy index, determined by the Fulton index [RV/(LV+S)](left panel) and the ratio of RV/BW (right panel) in Nor, SuHx3w, SuHx3w+Nor2w and SuHx3w+CHQ2w. * P<0.05, significantly different from Nor: #P<0.05, significantly different from SuHx3w+Nor2w.

FIG. 10. Chemical structure of ROC-325.

FIG. 11. ROC-325 triggers the formation of autophagosomes with undegraded cargo. MV4-11 cells were treated with 1 μM ROC-325 as indicated and visualized by transmission electron microscopy (TEM).

FIG. 12. Treatment with ROC-325 leads to lysosomal deacidification. Cells were treated with 1 μM ROC-325 for 6 h, stained with Lysosensor Green and propidium iodide (PI, nuclear counterstain) and then visualized by confocal microscopy. Loss of green fluorescence demonstrates lysosome deacidification.

FIG. 13. Chronic administration of ROC-325 is well tolerated. Mice were treated with vehicle, ROC-325, or HCQ as indicated daily for 42 consecutive days. No significant loss of mouse body weight occurred in any group. Mean±SD.

FIG. 14A-E. ROC-325 has promising therapeutic activity in models of PH. A-C: in vivo intact animal experiments (ROC-325 was administered daily at 10 mg/kg for 4w). (A) Representative record showing right ventricular pressure (RVP) in control and ROC-treated rats during normoxia. These data indicate that ROC has no effect on basal hemodynamics in control rats during hypoxia. (B) Representative record showing right ventricular pressure (RVP) in control rats, MCT-injected rats and MCT-injected rats treated with ROC. (C) Summarized data showing right ventricular systolic pressure (RVSP), a surrogate measure for pulmonary arterial systolic pressure (PVSP), in control, MCT-rats (treated with vehicle) and MCT-rats treated with ROC. These data indicate that ROC significantly inhibited the development of experimental PH. D-E: ex vivo experiment using isolated perfused/ventilated mouse lungs (ROC was applied via intrapulmonary perfusion for 5 min). (D) Representative record showing pulmonary arterial pressure (PAP) in isolated perfused/ventilated mouse lung before, during and after intrapulmonary arterial perfusion of 40 mM K+ (40K) in the absence or presence of ROC. (E) Summarized data showing 40K-induced increases in PAP before (control), during (ROC) and after (washout) application of ROC. These data indicate that ROC also acutely inhibits high K+-induced pulmonary vasoconstriction.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The term “compound” is used herein to describe any specific compound or bioactive agent disclosed herein, including any and all stereoisomers (including diasteromers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein. It is understood that the choice of substituents or bonds within a Markush or other group of substituents or bonds is provided to form a stable compound from those choices within that Markush or other group.

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Lower alkyl groups are those including at most 6 carbon atoms. Examples of alkyl groups include haloalkyl groups and hydroxyalkyl groups.

The term “aryl” as used herein includes carbocyclic aromatic rings or ring systems. Examples of aryl groups include phenyl, naphthyl, biphenyl, anthracenyl, phenanthracenyl, fluorenyl and indenyl. Aryl groups may be substituted or unsubstituted.

The term “autophagy” is used to describe a catabolic process in cells which involves the degradation of a cell's own components through lysosomes.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, delay in the onset of the disease, etc. Treatment also includes partial or total inhibition of autophagy and/or HDAC activity in a subject having a disease or condition that responds favorably to such inhibition.

As used herein, the term “prevention” includes either preventing the onset of a clinically evident disease or condition in individuals at risk. Also intended to be encompassed by this definition is the decrease of autophagy in cells to prevent the occurrence of a disease facilitated by autophagy. This includes prophylactic treatment of those having an enhanced risk of developing precancers and cancers facilitated by autophagy and/or aberrant HDAC activity. An elevated risk represents an above-average risk that a subject will develop a disease or condition involving autophagy (e.g., cancer), which can be determined, for example, through family history or the detection of genes causing a predisposition to autophagy-dependent disease.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent that will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the inhibition of autophagy by a detectable amount.

The term “subject” for purposes of treatment includes any human or animal subject who has a disorder facilitated by autophagy and/or aberrant HDAC activity. Such disorders include, but are not limited to cancer, pulmonary hypertension, diabetes, neurodegenerative disorders, aging, heart disease, rheumatoid arthritis, infectious diseases, etc. For methods of prevention the subject is any human or animal subject, and preferably is a human subject who is at risk of acquiring a disorder characterized by unwanted, rapid cell proliferation, such as cancer. The subject may be at risk due to exposure to carcinogenic agents, being genetically predisposed to disorders characterized by unwanted, rapid cell proliferation, and so on. Besides being useful for human treatment, the compounds of the present invention are also useful for veterinary treatment of mammals, including companion animals and farm animals, such as, but not limited to dogs, cats, horses, cows, sheep, and pigs. Preferably, subject means a human.

### DETAILED DESCRIPTION OF THE INVENTION

Autophagy is an evolutionarily conserved lysosomal protein degradation process. Aberrant autophagy plays a role in the pathogenesis of many health conditions including cancer, pulmonary hypertension, diabetes, neurodegenerative disorders, aging, heart disease, rheumatoid arthritis, infectious diseases, etc. The present invention relates to a new class of small-molecules having a quinoline or thioxanthenone (or similar) structure which function as autophagy inhibitors and/or histone deactylase inhibitors, and their use as therapeutics for the treatment of conditions characterized with aberrant autophagy activity and/or aberrant HDAC activity.

In a particular embodiment, compounds encompassed within Formula I are provided:

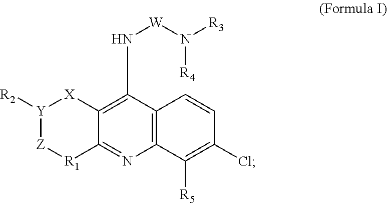

including pharmaceutically acceptable salts, solvates, and/or prodrugs thereof.

Formula I is not limited to a particular chemical moiety for W, X, Y, Z, R₁, R₂, R₃, R₄, and R₅. In some embodiments, the particular chemical moiety for W, X, Y, Z, R₁, R₂, R₃, R₄, and R₅ independently include any chemical moiety that permits the resulting compound to inhibit cellular autophagy. In some embodiments, the particular chemical moiety for W, X, Y, Z, R₁, R₂, R₃, R₄, and R₅ independently include any chemical moiety that permits the resulting compound to inhibit cellular HDAC activity. In some embodiments, the particular chemical moiety for W, X, Y, Z, R₁, R₂, R₃, R₄, and R₅ independently include any chemical moiety that permits the resulting compound to inhibit cellular autophagy and cellular HDAC activity. In some embodiments, the particular chemical moiety for W, X, Y, Z, R₁, R₂, R₃, R₄, and R₅ independently include any chemical moiety that permits the resulting compound to act as an effective therapeutic for treating conditions characterized with aberrant autophagy activity and/or aberrant HDAC activity (e.g., cancer, pulmonary hypertension, diabetes, neurodegenerative disorders, aging, heart disease, rheumatoid arthritis, infectious diseases, conditions and symptoms caused by a viral infection (e.g., COVID-19)).

Such embodiments are not limited to particular definition for W. In some embodiments, W is selected from

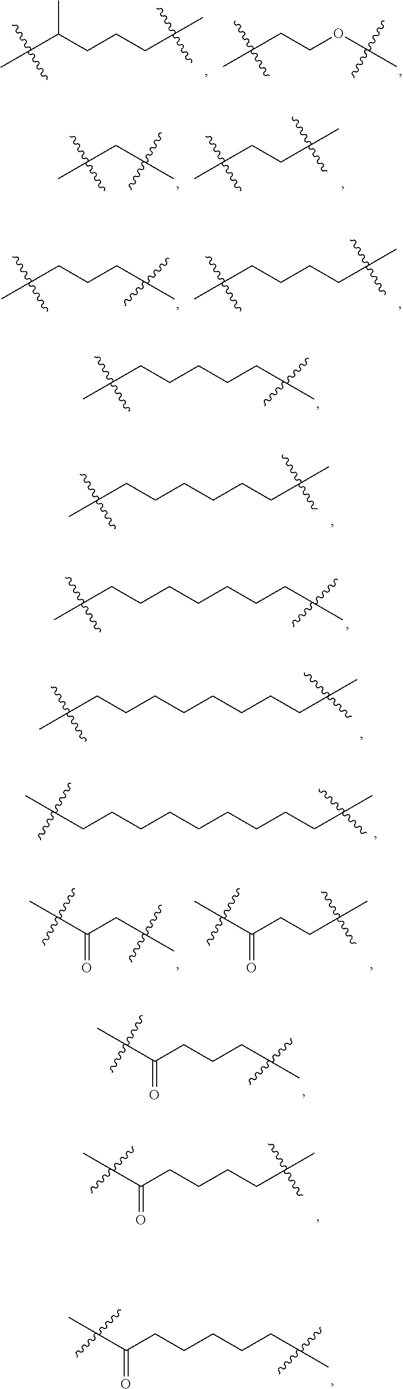

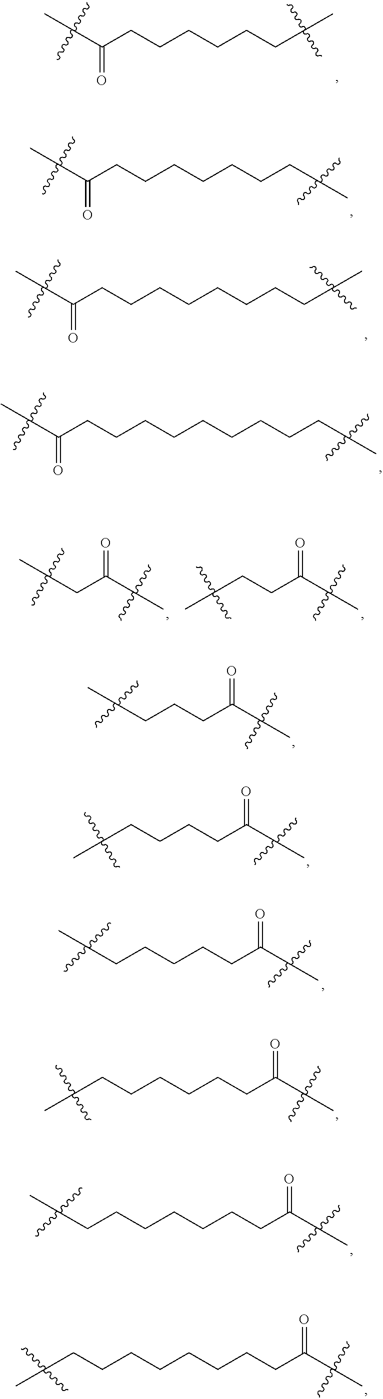

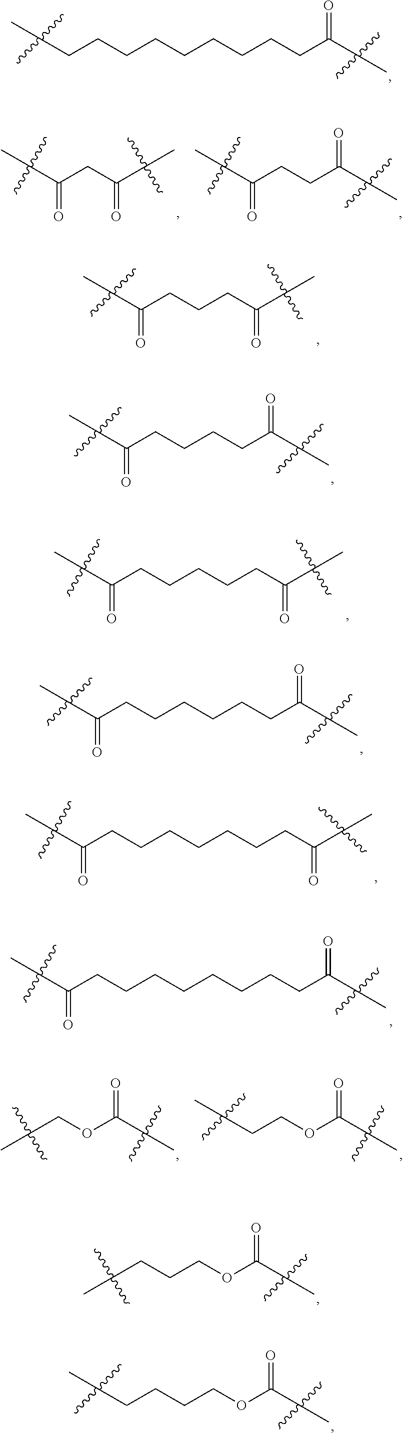

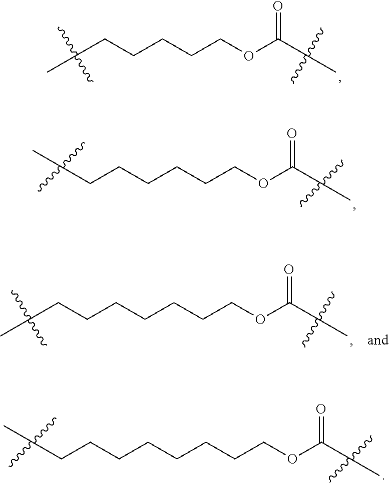 [SMILES: *CCCCCOC(*)=O ; *CCCCCCOC(*)=O ; *CCCCCCCOC(*)=O ; *CCCCCCCCOC(*)=O]

Such embodiments are not limited to a particular definition for

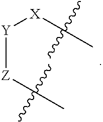

In some embodiments, each of X, Y, and Z are CH or N such that the resulting compound is represented by Formula II:

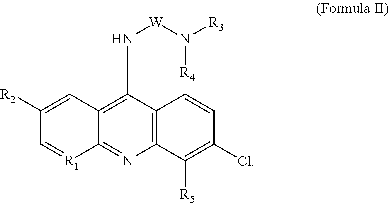

In some embodiments, each of X and Z are CH₂, N, and Y is CH such that the resulting compound is represented by Formula III:

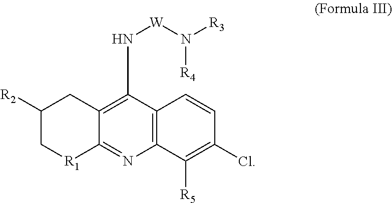

Such embodiments are not limited to a particular definition for R₁. In some embodiments, R₁ is CH₂, N-alkyl, aryl or NH or NCO—.

Such embodiments are not limited to a particular definition for R₂. In some embodiments, R₂ is methoxy, halogens, NH₂, alkyl, aryl, or hydrogen.

Such embodiments are not limited to a particular definition for R₃. In some embodiments, R₃ is selected from hydrogen, methyl, ethyl, Ph,

pyridine

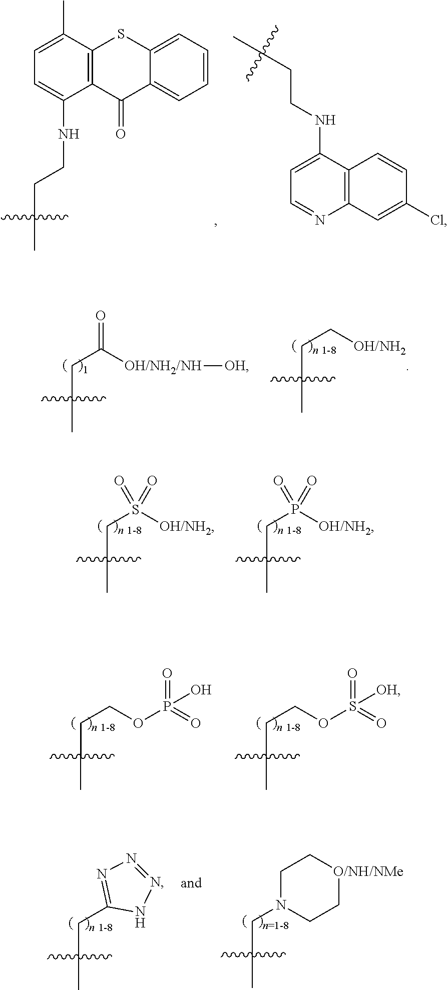

Such embodiments are not limited to particular definition for R₄.

In some embodiments, Ra is selected from hydrogen, methyl, ethyl, hydroxyl,

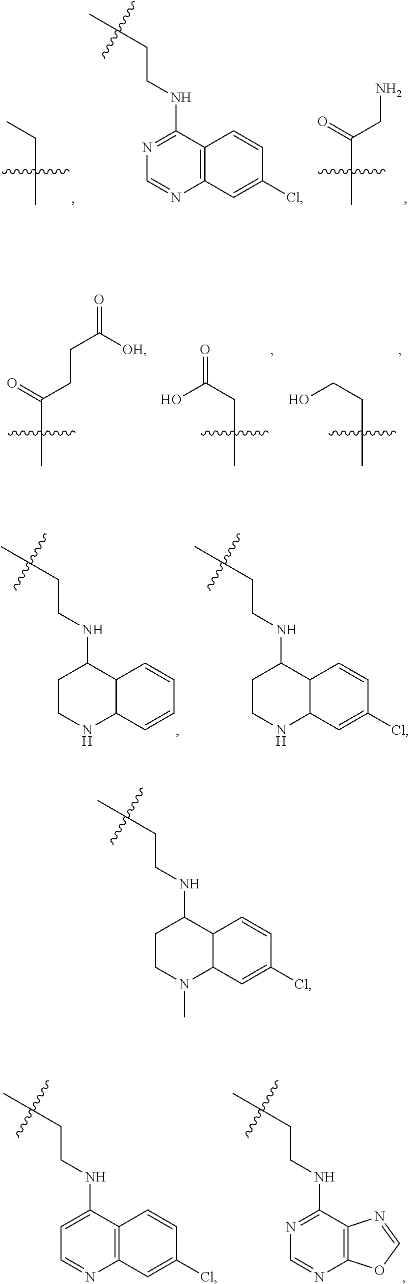

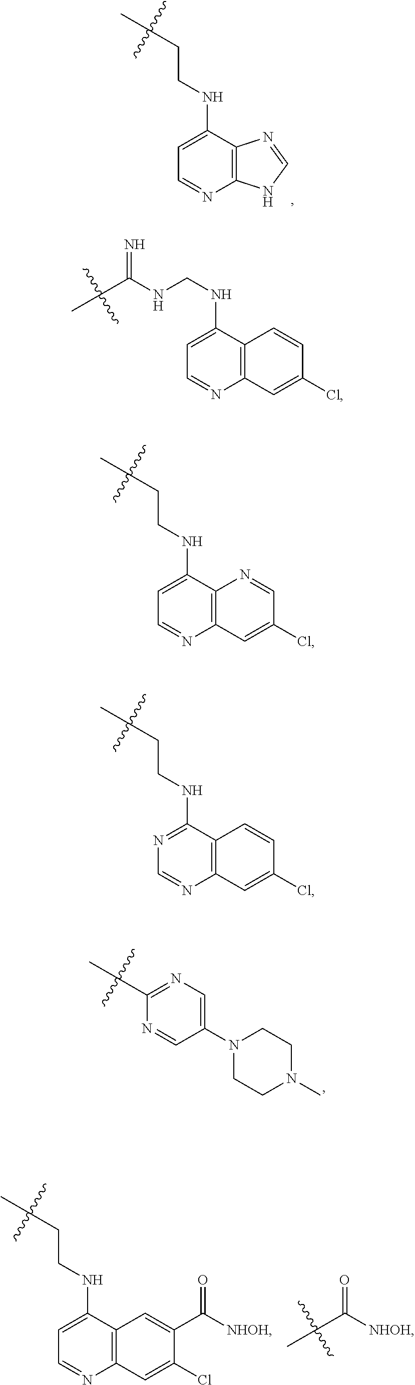

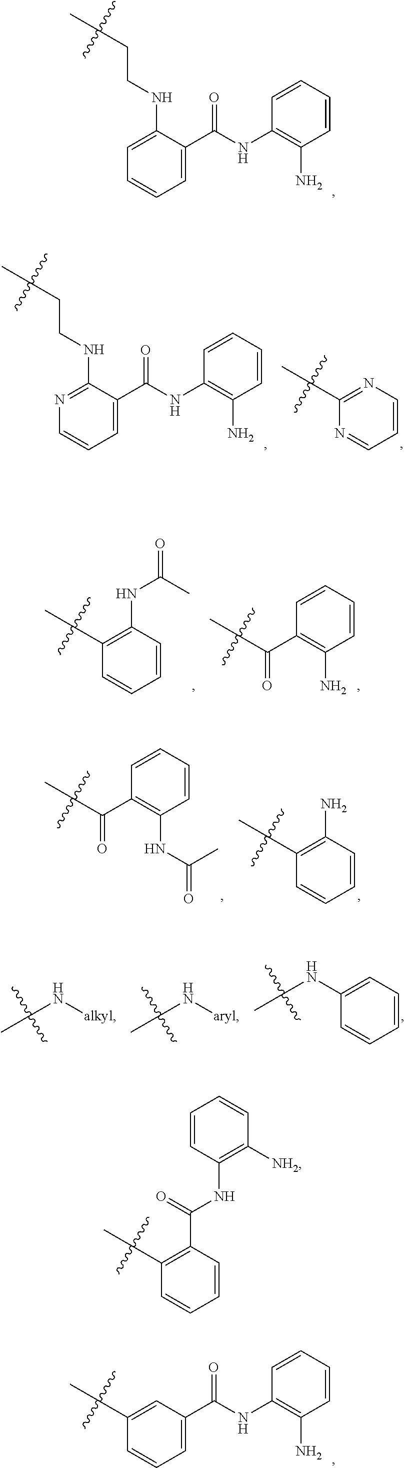

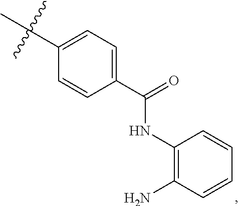 [SMILES: *c1ccc(C(=O)Nc2ccccc2N)cc1]

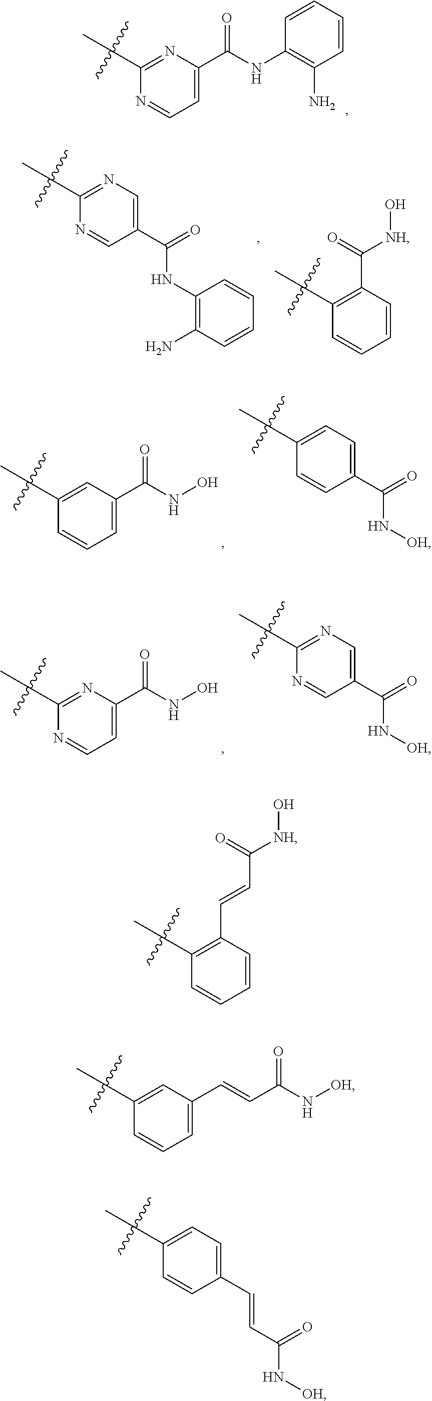

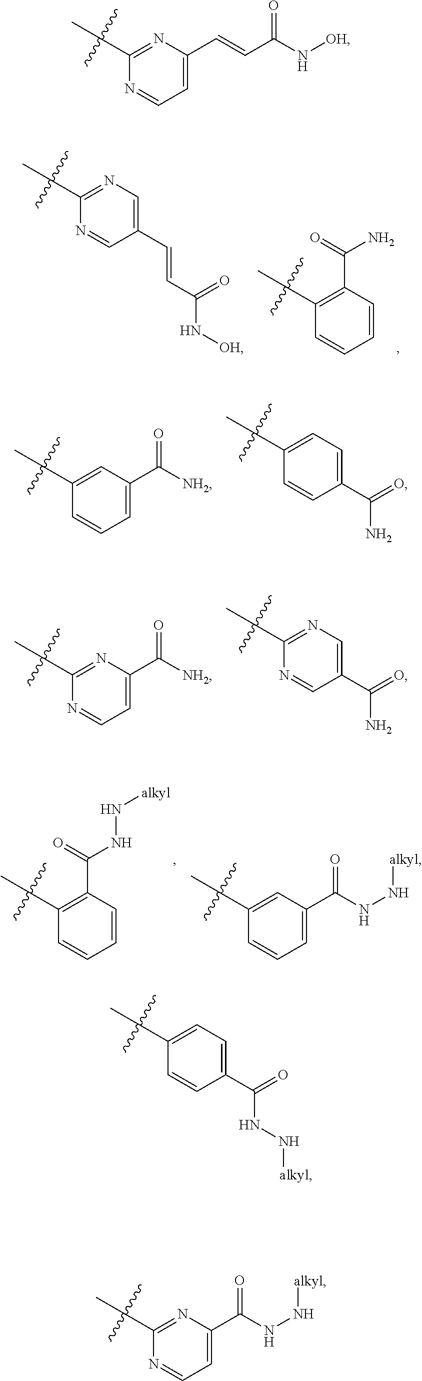

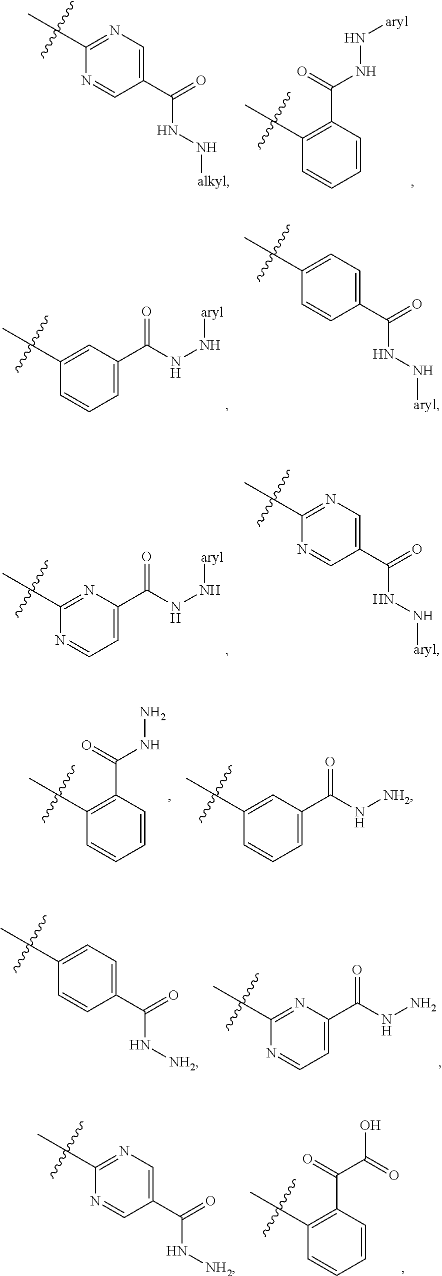

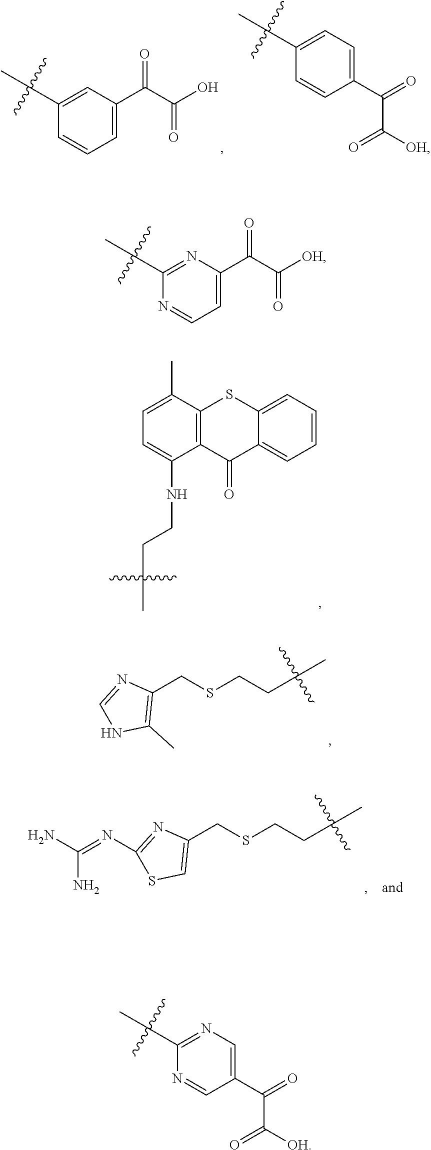

Such embodiments are not limited to particular definition for R₅. In some embodiments, R₅ is selected from hydrogen, methyl, and deuterated methyl (e.g., CD₃).

The invention further provides processes for preparing any of the compounds of the present invention.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions.

Autophagy is a type II programmed cell death and can initiate cell death in different circumstances. While autophagy plays an important and helpful role in many cells, in some cases it can result in undesirable damage. This damaging autophagic response have been attributed to many diseases and disorders such as neurodegenerative diseases (see, Fuchs Y, Steller H., Cell 2011:147: 742-58), cancer, liver diseases (see, Hidvegi et al., Science 2010); 329:229-32), cardiac diseases (see, Thapalia et al., Int J Clin Exp Pathol. 2014: 7 (12): 8322-41), metabolic syndromes (see, Codogno et al., Cell Metab 2010:11: 449-51), diabetes (see, Pareek et al., Curr Med Res Opin 2014:30: 1257-66), parasitic infection, aging (see, Rubinsztein et al., Cell 2011:146: 682-95), and inflammation (see, Virgin H W, Levine B. Nat Immunol 2009:10: 461-70).

A disease or condition that responds favorably to autophagy inhibition is a disease in which autophagy facilitates the progression of the disease or condition, and which is therefore treated by inhibiting autophagy. Examples of diseases or conditions which benefit from the inhibition of autophagy include cancer, diabetes, malaria, schistosomiasis, rheumatoid arthritis, antiphospolipid antibody syndrome, lupus, chronic urticaria, Sjögren's disease, reperfusion injury, and neurodegenerative diseases such as Huntington's disease, Alzheimer's disease, Parkinson's disease, and amyotrophic lateral sclerosis.

In some embodiments, the disease being treated is cancer. Cancer is a disease of abnormal and excessive cell proliferation. Cancer is generally initiated by an environmental insult, certain gene mutations or gene deletions, or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of malignant cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system.

Examples of types of cancer that can be treated using the compounds of the present invention include cancer is selected from the group consisting carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver, lung, nasopharyngeal, neck, ovary, pancreas, prostate, and stomach; leukemias, such as acute myclogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lynphoblastic leukemia, lymphocytic leukemia, megakaryocytic leukemia, micromyeloblastic leukemia, monocytic leukemia, neutrophilic leukemia and stem cell leukemia: benign and malignant lymphomas, particularly Burkitt's lymphoma, Non-Hodgkin's lymphoma and B-cell lymphoma: benign and malignant melanomas: myeloproliterative diseases: sarcomas, particularly Ewing's sarcoma, hemangiosarcoma, Kaposi's sarcoma, liposarcoma, myosarcomas, peripheral neuroepithelioma, and synovial sarcoma: tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas): germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma: mixed types of neoplasias, particularly carcinosarcoma and Hodgkin's disease; and tumors of mixed origin, such as Wilms' tumor and teratocarcinomas, among others. Certain epithelial tumors including ovarian, breast, colon, head and neck, medulloblastoma and B-cell lymphoma, among others are shown to exhibit increased autophagy and are principal target cancers for the autophagy inhibitors of the present invention in some embodiments, while in other embodiments the cancer is leukemia or acute myeloid leukemia.

In some embodiments, the method further comprises administration of an additional anticancer agent. An additional anticancer agent is known anticancer agent that can be co-administered with one or more compounds of the present invention for cancer treatment. Co-administration can either be simultaneous, or proximal in time. Examples of anticancer agents include angiogenesis inhibitors such as angiostatin KI-3, DL-α-difluoromethyl-omithine, endostatin, fumagillin, genistein, minocycline, staurosporine, and (+)-thalidomide: DNA intercalating or cross-linking agents such as bleomycin, carboplatin, carmustine, chlorambucil, cyclophosphamide, cisplatin, phenanthriplatin, meiphalan, mitoxantrone, and oxaliplatin: DNA synthesis inhibitors such as methotrexate, 3-Amino-1,2,4-henzotriazine 1,4-dioxide, aminopterin, cytosine-D-arabinofuranoside, 5-Fluoro-5′-deoxyuridine, S-Fluorouracil, gaciclovir, hydroxyurea, and mitomycin C: DNA-RNA transcription regulators such as actinomycin D, daunorubicin, doxorubicin, homoharringtonine, and idarubicin: enzyme inhibitors such as S (+)-camptothecin, curcumin, (−)-deguelin, 5,6-dichlorobenz-imidazole 1-β-D-ribofuranoside, etoposine, formestane, fistriecin, hispidin, cyclocreatine, mevinolin, trichostatin A, tyrophostin AG 34, and tyrophostin AG 879, Gene Regulating agents such as 5-aza-2′-deoxycitidine, azacitidine, decitabine, cholecalciferol, 4-hydroxytamoxifen, melatonin, mifepristone, raloxifene, all trans-retinal, all trans retinoic acid, 9-cis-retinoic acid, retinol, tamoxifen, and troglitazone: Microtubule Inhibitors such as colchicine, dolostatin 15, nocodazole, paclitaxel, podophyllotoxin, rhizoxin, vinblastine, vincristine, vindesine, and vinorelbine; and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-α, rapamycin, thapsigargin, and bikunin, and derivatives thereof.

Candidate agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers (see, for instance, Polin et al., Investig. New Drugs, 15:99-108 (1997)). Results are typically compared between control animals treated with candidate agents and the control littermates that received a vehicle that does not contain the test agent. Transgenic animal models are also available and are commonly accepted as models for human disease (see, for instance. Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof.

Another aspect of the invention provides a method of inhibiting autophagy in cells of a subject, comprising contacting the cells with an effective amount of a compound recited herein.

Autophagy is a highly regulated process of biological systems that plays a normal part in cell growth development and homeostasis helping to maintain a balance between the synthesis, degradation, and subsequent recycling of cellular products. It is a major mechanism by which a cell allocates nutrients from unnecessary processes to more-essential processes.

A number of autophagic processes occur in nature, all of which have the degradation of intracellular components via the lysosome as a common feature. A well-known mechanism of autophagy involves the formation of a membrane around a targeted region of a cell, separating the contents from the rest of the cytoplasm. The resultant vesicle then fuses with a lysosome which subsequently degrades the contents.

Autophagy consists of the sequestration of organelles and proteins in autophagic vesicles (AV) and degradation of this cargo through lysosomal fusion. Autophagy allows tumor cells to survive metabolic and therapeutic stresses. Multiple publications by those skilled in the art indicate therapy-induced autophagy is a key resistance mechanism to many anti-cancer agents.

The present invention includes methods of inhibiting autophagy in a cell, particularly the cell of a subject. The methods can be used to inhibit autophagy in a cell in various environments. For example, in some embodiments, the cells are contacted in vivo, while in other embodiments the cells are contacted in vitro or ex vivo. The resulting inhibition of authophagy can be monitored or applied in the cell to evaluate effectiveness of the agent, effect a favorable result such as treatment of a disease or condition involving autophagy, or to study the effect of inhibition of autophagy in the cell.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragées, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragée-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragée cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragée cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragée coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

Experiments conducted during the course of developing embodiments for the present invention designed, synthesized and biologically evaluated compounds having a tetrahydroacridine (or similar) structure, an acridine (or similar) structure, a benzonaphthyridine (or similar) structure, or a tetrahydrobenzonaphthyridine (or similar) structure as autophagy inhibitors and/or histone deactylase (HDAC) inhbitors, and their potential for use as therapeutics against conditions characterized with aberrant autophagy activity and/or aberrant HDAC activity (e.g., cancer, pulmonary hypertension, diabetes, neurodegenerative disorders, aging, heart disease, rheumatoid arthritis, infectious diseases, etc.).

### Example II

Idiopathic pulmonary arterial hypertension (PAH) is a fatal and progressive disease with a very poor prognosis. The estimated median survival is only 2.8 years with a 5-year survival rate of 34% (1-3). Excessive pulmonary vascular remodeling, sustained vasoconstriction, in situ thrombosis and increased pulmonary vascular wall stiffness all directly result in elevated PVR (pulmonary vascular resistance) and pulmonary arterial pressure (PAP) in patients with PAH (2-8). The elevated PVR/PAP increase RV (right ventricular) afterload and induce RV hypertrophy, which can progress to right heart failure and death. The dismal survival prospects for those diagnosed with PAH highlights the imperative need to develop precision therapies with improved safety profiles based on our understanding of the specific aberrations that drive disease progression.

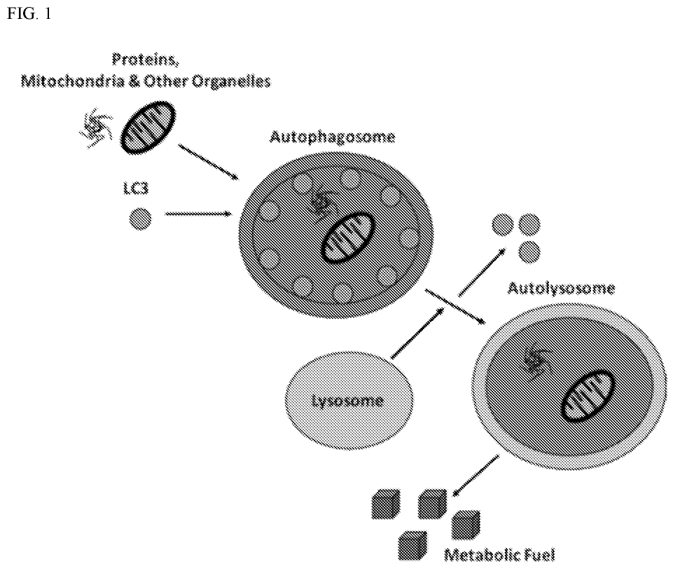

Autophagy is an evolutionarily conserved lysosomal-mediated degradation process that facilitates the turnover of organelles and selected long-lived proteins. Outside of its role in maintaining cellular homeostasis, autophagy is frequently stimulated in response to stress conditions such as those imposed by oxidative stress, nutrient deprivation, and microenvironmental disruption. In this capacity, autophagy serves as a mechanism to generate alternative sources of metabolic fuel that promote survival when preferred energy generating pathways are impaired (FIG. 1) (9). Over the past decade, stress-induced autophagy has emerged as an important driver of PH pathogenesis. Alveolar hypoxia-induced pulmonary vasoconstriction (HPV) directs blood flow in the lungs in a targeted manner to maximize venous blood oxygenation in the pulmonary circulation. Sustained HPV in patients with obstructive lung diseases and mountain sickness can stimulate hypoxia-induced autophagy. Persistent autophagy in the lungs can result in chronic pulmonary vasoconstriction, vascular remodeling, and ultimately, chronic PH. Accordingly, we and others have demonstrated that disrupting autophagy is a promising approach to alleviate key aspects of PH pathogenesis (10-13).

The FDA approved anti-malarial drugs chloroquine (CQ) and hydroxychloroquine (HCQ) are members of a very small group of drugs known to inhibit autophagy through the disruption of lysosomal function (14,15). These specific properties of CQ/HCQ spurred numerous preclinical investigations focused on repurposing CQ/HCQ as an autophagy inhibitor for many indications including cancer and PH (10,13,16,17). Based on the positive impact of HCQ in this scenario, we initiated a Phase I clinical trial to investigate the safety and preliminary efficacy of the addition of HCQ to the histone deacetylase inhibitor vorinostat (14). Our trial, like many other CQ/HCQ repurposing studies, yielded modest clinical benefit characterized by partial responses and stable disease, but few sustained complete responses. Despite the initial subdued clinical results reported from these trials, there is maintained enthusiasm for autophagy inhibition as therapeutic approach for two overarching reasons. First, it is unlikely that the maximum tolerated dose (MTD) of HCQ is sufficient to completely inhibit autophagy in humans. In our Phase I trial, pharmacokinetic (PK) analyses showed that the Cmax of HCQ in the MTD cohort was less than 3 μM. This is significantly below the concentration of CQ/HCQ that is required to completely inhibit autophagic activity in most published preclinical studies. Residual autophagic activity may have been a factor that limited efficacy in these studies. Second, despite their prevalent use as autophagy inhibitors, the reality is that CQ/HCQ are very old drugs that were not designed to inhibit autophagy. Their continued use for this purpose is driven by the lack of better alternatives. New autophagy inhibitors with increased potency and more favorable therapeutic indices are clearly needed to definitively determine the efficacy of this approach, but none have been tested in humans to date.

Following the completion of the initial HCQ cancer clinical trials, the demand for superior autophagy inhibitors grew exponentially. However, no comprehensive structure activity relationship (SAR) analyses on CQ/HCQ had been conducted to date and it was therefore unclear which specific structural modifications to these agents may result in increased anti-autophagic potency or efficacy. Polyvalent molecules have the potential to yield nonlinear, multifold potency against their respective targets compared to their corresponding monomers (18,19). With the goal of generating new autophagy inhibitors with better potency, tolerability, and efficacy than HCQ, we used logical medicinal chemistry approaches to generate a series of new dimeric compounds containing modified core elements of HCQ and the anti-schistosomal drug lucanthone, which we previously reported inhibits autophagy (20). ROC-325 emerged as an initial lead compound that contained structural motifs of both HCQ and lucanthone. ROC-325 is orally available, well tolerated, inhibits autophagy at significantly lower doses and exhibits significantly superior single agent anticancer activity against a broad range of tumor types compared to HCQ (21,22). Preliminary studies in models of PH also revealed that ROC-325 has very promising therapeutic effects. A major goal is to generate novel derivatives of ROC-325 with higher potency and optimize pharmacological properties, test their safety, efficacy, and mechanism of action in models of PH, and identify lead compounds for clinical testing. SAR studies on our initial compound series have identified rational strategies to accomplish this major goal.

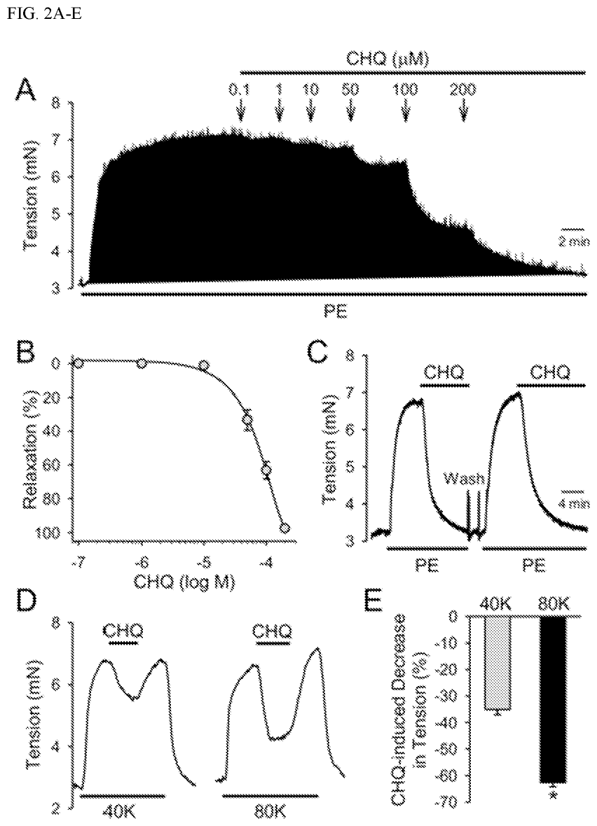

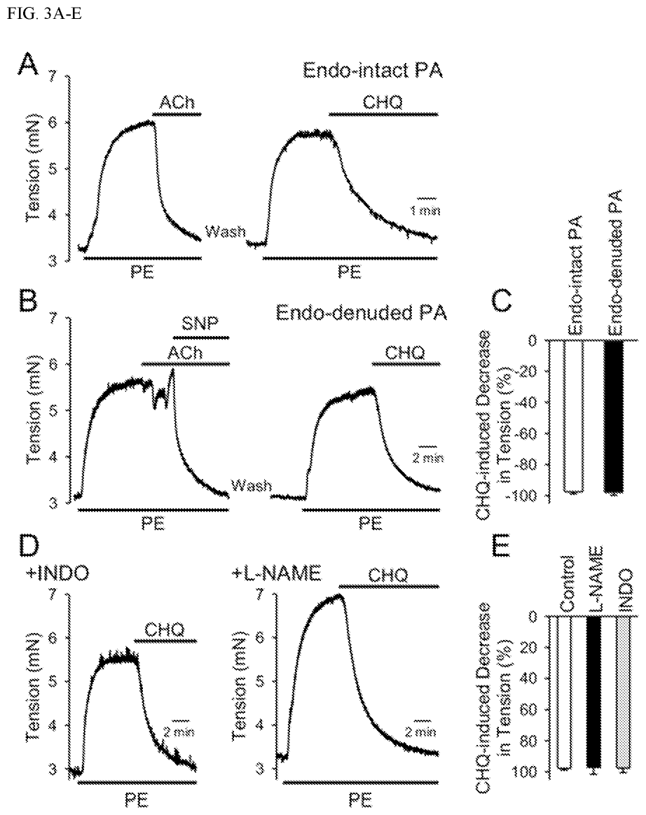

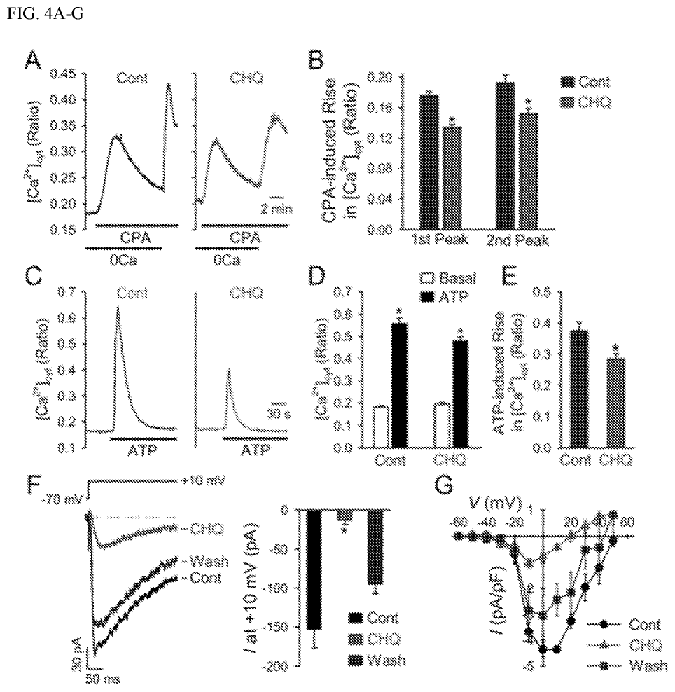

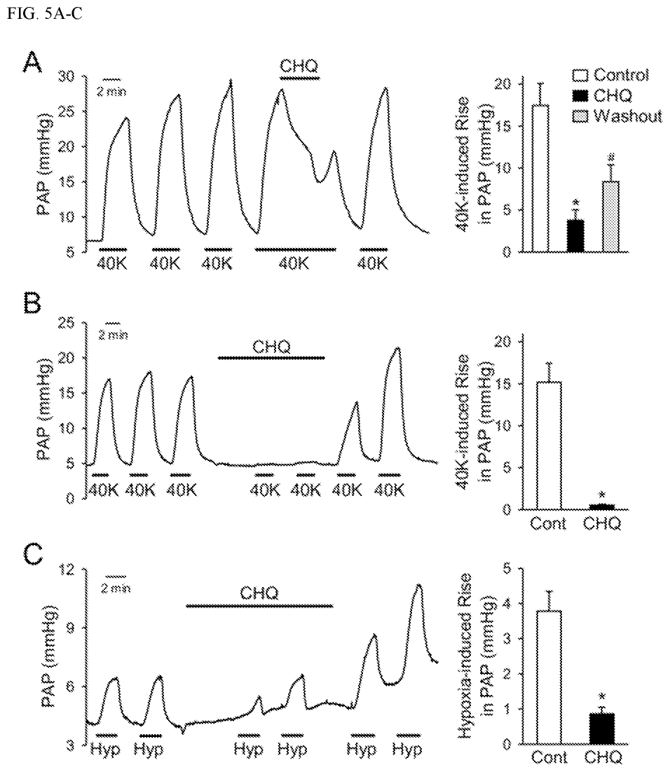

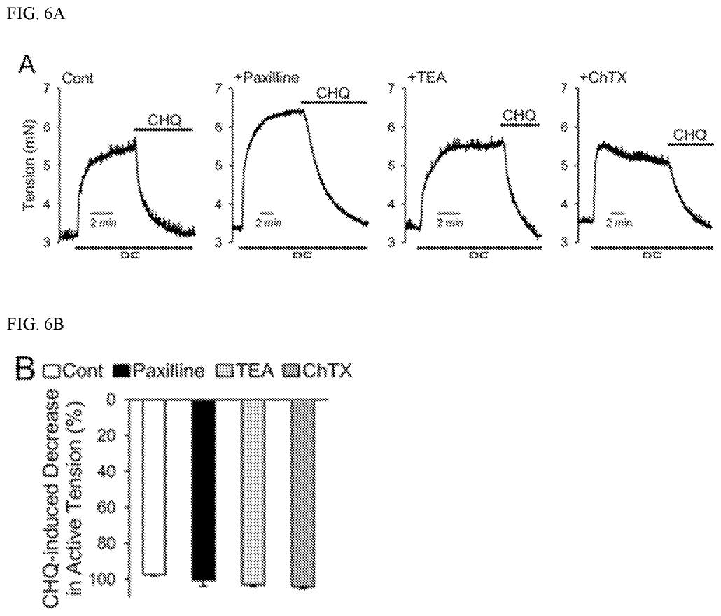

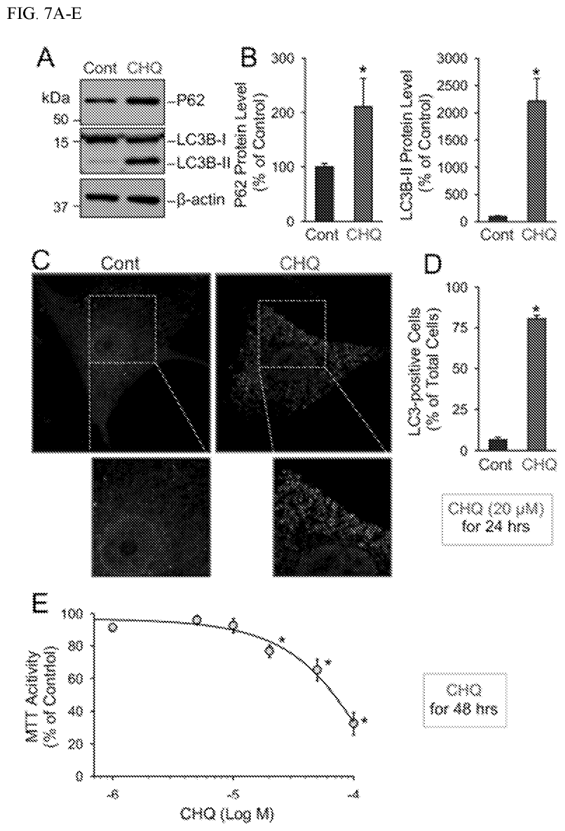

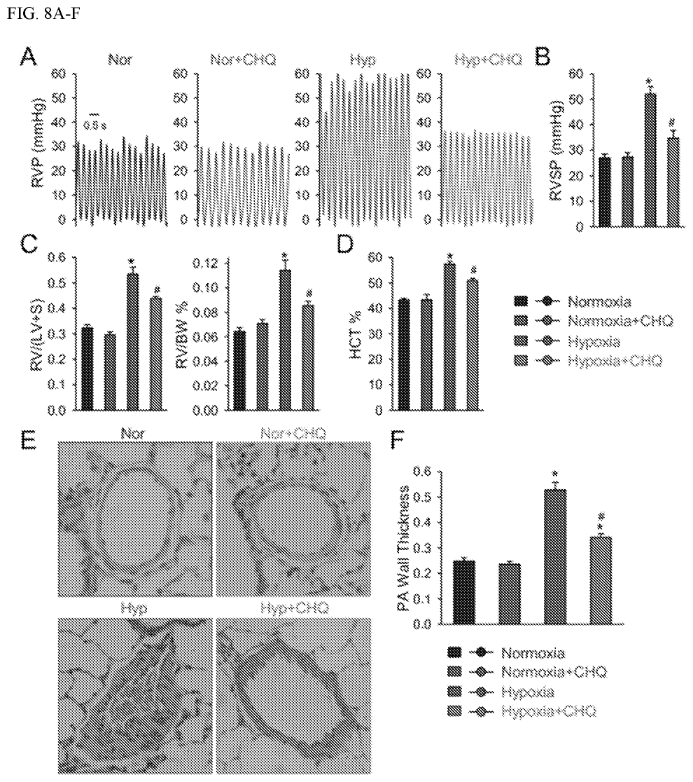

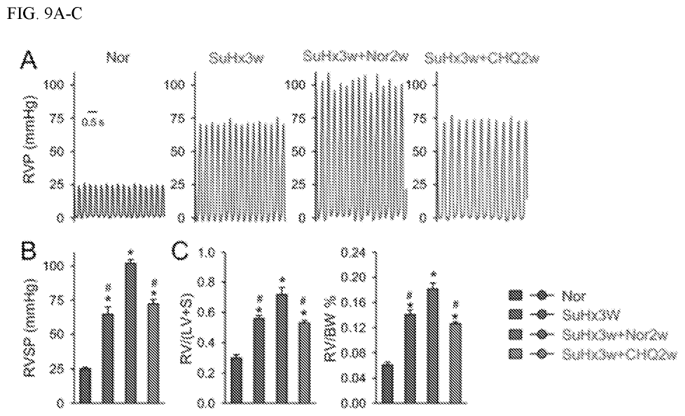

Due to the key role of autophagy in the pathogenesis of many diseases, we (the inventors) were very interested in exploring the benefit of autophagy inhibition for other indications beyond cancer. Based on evidence from multiple studies reporting that autophagy contributed to the development and progression of PH, experiments were conducted to rigorously test the potential therapeutic benefit of autophagy inhibition using chloroquine (CHQ/CQ) as a pharmacological autophagy inhibitor. Such experiments first quantified the vasorelaxant effect on rings of rat PA (FIG. 2). These experiments showed that CHQ decreased isometric tension in isolated PA rings constricted with 1 μM phenylephrine (PE). Similarly. CHQ decreased active tension in PA rings constricted with 40 mM (40K) or 80 mM (80K) K+-containing solution before, during and after application of 200 μM CHQ. We next evaluated the potential role of the endothelium on the vasorelaxant effect of CHQ on isolated rat PA (FIG. 3). These studies showed that CHQ (200 μM) induced decreases in active tension in endothelium-intact or endothelium-denuded rat PA rings pre-constricted with 1 μM PE. Thus, the vasorelaxant effect of CHQ is endothelium independent. We next assessed the impact of CHQ treatment on Ca2+ dynamics in PASMC (FIG. 4). This series of experiments established that CHQ inhibits store-operated Ca2+ entry (ROCE) and reduces whole-cell Ca2+ currents in human PASMC. We next tested the ability of CHQ to diminish vasoconstriction caused by hypoxia or high K+ (FIG. 5). Our studies showed that CHQ could antagonize the 40K-induced increases in PAP in isolated perfused/ventilated lungs. Collectively, our data demonstrate that CHQ attenuates pulmonary vasoconstriction induced by high K+ concentrations or hypoxia. We next sought to determine whether blockade of K+ channels would impair the pulmonary vasodilation effects of CHQ (FIG. 6). These assays showed that pharmacological inhibition of K+ channels does not affect the ability of CHQ to induce pulmonary vasodilation. We subsequently evaluated the effects of CHQ on autophagy and cell proliferation in human PASMC (FIG. 7). This series of assays demonstrated that treatment with CHQ inhibited autophagy as shown by the effects of drug treatment on p62 and LC3B. CHQ also exhibited dose-dependent anti-proliferative effects on human PASMC. We next investigated the ability of CHQ to inhibit the development of HPH in rats (FIG. 8). Right ventricular systolic pressure (RVSP), a surrogate measure of pulmonary arterial systolic pressure, was quantified in rats exposed to normoxia alone (Nor) or with CHQ (50 mg/kg/day. Nor+CHQ) treatment and rats exposed to hypoxia (Hyp: 10% 02 for 21 days) or with CHQ (Hyp+CHQ) treatment. These data showed that CHQ significantly reduced RVSP. Additionally. CHQ significantly reduced the RV hypertrophy, as determined by the Fulton index, and reduced PA wall thickness. Finally, we tested the ability of CHQ to antagonize progression of established PH in the SuHx-PH rat model (FIG. 9). Rats were given a subcutaneous (s.c.) injection of 20 mg/kg SU5416 or vehicle, followed by hypoxia (SuHx) for 3 weeks (10% 02) and normoxia for an additional 2 weeks to elicit severe PH. After 3 weeks of hypoxia, the SuHx rats were divided into three groups: hypoxia with no intervention (SuHx3w), vehicle treatment (SuHx3w+Nor2w) and treatment with CHQ (50 mg/kg/day, SuHx3w+CHQ2w). The normoxic control group was exposed to room air only (21% 02) for a total of 5 weeks (Nor). Our collective data demonstrated that CHQ provided significant therapeutic benefit against established PH progression and partially reversed established PH.

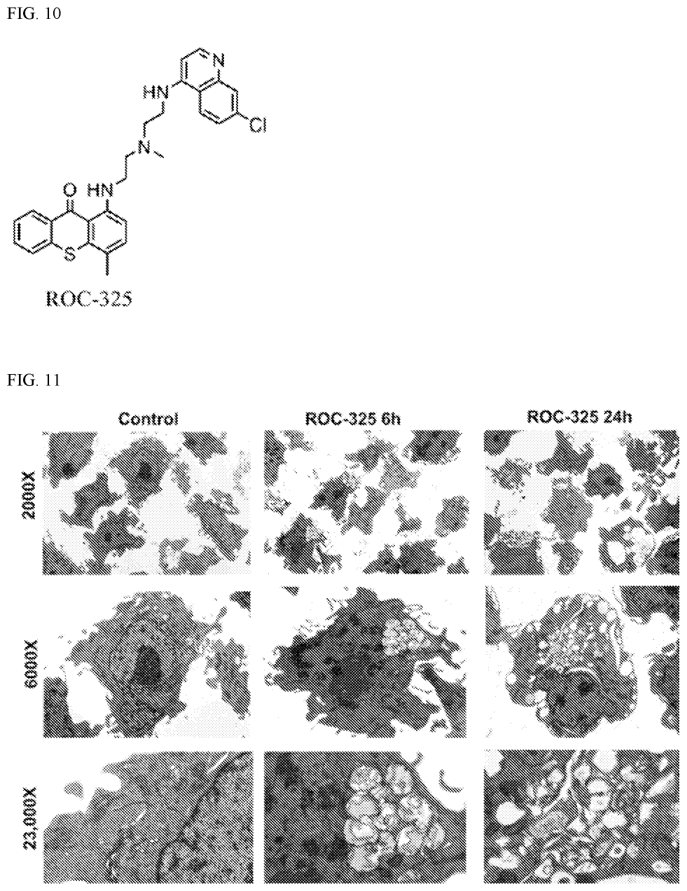

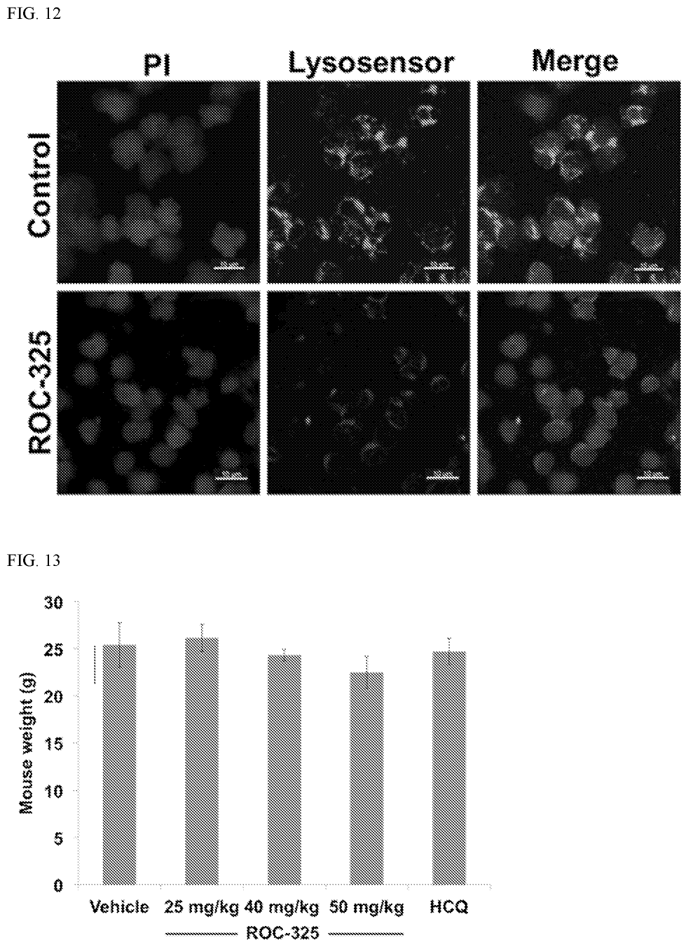

Our collective experience with CQ/HCQ in PH and cancer established proof of concept that therapeutic autophagy inhibition is a promising approach for diseases where autophagy contributes to disease pathogenesis. However, better drugs are needed to effectively target this pathway clinically. As mentioned earlier, PK analyses of the Cmax of HCQ at the MTD (600 mg/day) in our Phase I trial illuminated the need for novel autophagy inhibitors that more potently disrupt autophagic degradation at tolerable doses in humans (14). We next directed our efforts toward designing a series of novel lysosomal autophagy inhibitors with superior potency, tolerability, and efficacy (20.26). Initial preclinical studies identified ROC-325 as a lead agent for further investigation as it demonstrated approximately 10-fold greater potency than HCQ and is an orally bioavailable compound (FIG. 10). Rigorous assays showed that ROC-325 triggers all of the hallmark features of autophagy inhibition. Example data from our published studies are shown here. Transmission electron microscopy (TEM) analyses demonstrated that treatment of leukemia cells with ROC-325 triggered the accumulation of autophagosomes with undegraded cargo (FIG. 11). The majority of lysosomal proteases that control the autophagy-mediated degradation of proteins and organelles require a strict acidic pH microenvironment for their enzymatic activity. Based on this, lysosomal acidity is commonly used as a marker of autophagy status. We treated leukemia cells with ROC-325 and used LysoSensor Green to evaluate the effects of drug treatment on lysosomal pH. Confocal microscopy and FACS-based quantification of LysoSensor Green fluorescence both showed that ROC-325 stimulated lysosomal deacidification (FIG. 12, P<0.01). Additional assays showed that ROC-325 stimulated the autophagy marker LC3B along with accumulation of p62, whose turnover is specifically mediated by autophagy (22). Treatment of normal human CD34+ bone marrow (BM) cells and renal proximal tubal endothelial cells (RPTEC) with ROC-325 demonstrated strong therapeutic selectivity (22,26). Accordingly, chronic oral daily administration of ROC-325 to mice for 42 days confirmed its tolerability (FIG. 13) (26).

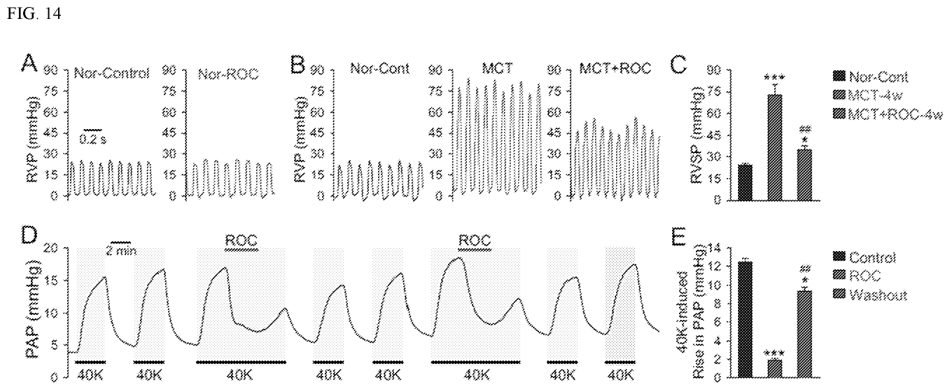

Finally, we tested the therapeutic benefit of ROC-325 in models of experimental PH (FIG. 14). ROC-325 was administered to rats daily at 10 mg/kg for 4w under normoxic and hypoxic conditions. Our data indicate that ROC-325 has no effect on basal hemodynamics in control rats during hypoxia. Quantification of RVSP in control rats, MCT-injected rats and MCT-injected rats treated with ROC-325 demonstrated that ROC-325 significantly inhibited the development of experimental PH. Additional ex vivo experiments using isolated perfused/ventilated mouse lungs (ROC-325 was applied via intrapulmonary perfusion for 5 min) showed that ROC-325 also acutely inhibits high K+-induced pulmonary vasoconstriction. Our collective preliminary studies strongly support the optimization of the potency and pharmacological properties of ROC-325 to identify lead compounds for eventual clinical testing in patients with PH.

### INCORPORATION BY REFERENCE

The entire disclosure of each of the patent documents and scientific articles referred to herein is incorporated by reference for all purposes. The following references are herein incorporated by reference in their entireties:
1. Hassoun P M, Mouthon L, Barbera J A, Eddahibi S, Flores S C, Grimminger F, et al. Inflammation, growth factors, and pulmonary vascular remodeling. Journal of the American College of Cardiology 2009: 54 (1Suppl): $10-9 doi 10.1016/j.jacc.2009.04.006.
2. Morrell N W, Adnot S, Archer S L, Dupuis J, Jones P L, MacLean M R, et al. Cellular and molecular basis of pulmonary arterial hypertension. Journal of the American College of Cardiology 2009: 54 (1 Suppl): S20-31 doi 10.1016/j.jacc.2009.04.018.
3. Sutendra G, Michelakis E D. The metabolic basis of pulmonary arterial hypertension. Cell Metab 2014: 19 (4): 558-73 doi 10.1016/j.cmet.2014.01.004.
4. Pietra G G, Edwards W D, Kay J M, Rich S, Kernis J, Schloo B, et al. Histopathology of primary pulmonary hypertension. A qualitative and quantitative study of pulmonary blood vessels from 58 patients in the National Heart, Lung, and Blood Institute, Primary Pulmonary Hypertension Registry. Circulation 1989: 80 (5): 1198-206.
5. Tuder R M, Archer S L, Dorfmuller P, Erzurum S C, Guignabert C, Michelakis E, et al. Relevant issues in the pathology and pathobiology of pulmonary hypertension. Journal of the American College of Cardiology 2013: 62 (25 Suppl): D4-12 doi 10.1016/j.jacc.2013.10.025.
6. Rabinovitch M. Molecular pathogenesis of pulmonary arterial hypertension. The Journal of clinical investigation 2012: 122 (12): 4306-13 doi 10.1172/JCI60658.
7. Kuhr F K, Smith K A, Song M Y, Levitan I, Yuan J X. New mechanisms of pulmonary arterial hypertension: role of Ca2+ signaling. American journal of physiology Heart and circulatory physiology 2012: 302 (8): H1546-62 doi 10.1152/ajpheart.00944.2011.
8. Sutendra G, Michelakis E D. Pulmonary arterial hypertension: challenges in translational research and a vision for change. Sci Transl Med 2013: 5 (208): 208sr5 doi 10.1126/scitranslmed.3005428.
9. Wang Y, Xia Y, Lu Z. Metabolic features of cancer cells. Cancer Commun (Lond) 2018: 38 (1): 65 doi 10.1186/s40880-018-0335-7.
10. Wu K, Zhang Q, Wu X, Lu W, Tang H, Liang Z, et al. Chloroquine is a potent pulmonary vasodilator that attenuates hypoxia-induced pulmonary hypertension. Br J Pharmacol 2017: 174 (22): 4155-72 doi 10.1111/bph. 13990.
11. Zhang Q, Tsuji-Hosokawa A, Willson C, Watanabe M, Si R, Lai N, et al. Chloroquine differentially modulates coronary vasodilation in control and diabetic mice. Br J Pharmacol 2020: 177 (2): 314-27 doi 10.1111/bph.14864.
12. Ornatowski W, Lu Q, Yegambaram M, Garcia A E, Zemskov E A, Maltepe E, et al. Complex interplay between autophagy and oxidative stress in the development of pulmonary disease. Redox Biol 2020:36:101679 doi 10.1016/j.redox.2020.101679.
13. Long L, Yang X, Southwood M, Lu J, Marciniak S J, Dunmore B J, et al. Chloroquine prevents progression of experimental pulmonary hypertension via inhibition of autophagy and lysosomal bone morphogenetic protein type II receptor degradation. Circ Res 2013: 112 (8): 1159-70 doi 10.1161/CIRCRESAHA.111.300483.
14. Mahalingam D, Mita M, Sarantopoulos J, Wood L, Amaravadi R K, Davis L E, et al. Combined autophagy and HDAC inhibition: a phase I safety, tolerability, pharmacokinetic, and pharmacodynamic analysis of hydroxychloroquine in combination with the HDAC inhibitor vorinostat in patients with advanced solid tumors. Autophagy 2014: 10 (8): 1403-14 doi 10.4161/auto.29231.
15. Chude C I, Amaravadi R K. Targeting Autophagy in Cancer: Update on Clinical Trials and Novel Inhibitors. Int J Mol Sci 2017: 18 (6) doi 10.3390/ijms 18061279.
16. Rao R, Balusu R, Fiskus W, Mudunuru U, Venkannagari S, Chauhan L, et al. Combination of pan-histone deacetylase inhibitor and autophagy inhibitor exerts superior efficacy against triple-negative human breast cancer cells. Mol Cancer Ther 2012; 11 (4): 973-83 doi 10.1158/1535-7163.MCT-11-0979.
17. Amaravadi R K, Yu D, Lum J J, Bui T, Christophorou M A, Evan G I, et al. Autophagy inhibition enhances therapy-induced apoptosis in a Myc-induced model of lymphoma. J Clin Invest 2007: 117 (2): 326-36.
18. Chow L M, Chan T H. Novel classes of dimer antitumour drug candidates. Current pharmaceutical design 2009: 15 (6): 659-74.
19. Hadden M K, Blagg B S. Dimeric approaches to anti-cancer chemotherapeutics. Anti-cancer agents in medicinal chemistry 2008: 8 (7): 807-16.
20. Carew J S, Espitia C M, Esquivel J A, 2nd, Mahalingam D, Kelly K R, Reddy G, et al. Lucanthone is a novel inhibitor of autophagy that induces cathepsin D-mediated apoptosis. J Biol Chem 2011: 286 (8): 6602-13 doi 10.1074/jbc.M110.151324.
21. Carew S S, Espitia C M, Zhao W, Han Y, Visconte V, Phillips J, et al. Disruption of Autophagic Degradation with ROC-325 Antagonizes Renal Cell Carcinoma Pathogenesis. Clin Cancer Res 2017:23:2869-79.
22. Nawrocki S T, Han Y, Visconte V, Przychodzen B, Espitia C M, Phillips J, et al. The novel autophagy inhibitor ROC-325 augments the antileukemic activity of azacitidine. Leukemia 2019: 33 (12): 2971-4 doi 10.1038/s41375-019-0529-2.
23. Carew J S, Nawrocki S T, Kahue C N, Zhang H, Yang C, Chung L, et al. Targeting autophagy augments the anticancer activity of the histone deacetylase inhibitor SAHA to overcome Bcr-Abl-mediated drug resistance. Blood 2007: 110 (1): 313-22.
24. Carew J S, Medina E C, Esquivel J A, 2nd, Mahalingam D, Swords R. Kelly K, et al. Autophagy inhibition enhances vorinostat-induced apoptosis via ubiquitinated protein accumulation. J Cell Mol Med 2010: 14 (10): 2448-59 doi JCMM832 [pii]10.1111/j.1582-4934.2009.00832.x.
25. Carew J S, Nawrocki S T, Cleveland J L. Modulating autophagy for therapeutic benefit. Autophagy 2007: 3 (5): 464-7 doi 4311 [pii].
26. Carew J S, Espitia C M, Zhao W, Han Y, Visconte V, Phillips J, et al. Disruption of Autophagic Degradation with ROC-325 Antagonizes Renal Cell Carcinoma Pathogenesis. Clin Cancer Res 2017: 23 (11): 2869-79 doi 10.1158/1078-0432.CCR-16-1742.
27. Kaufmann A M, Krise J P. Lysosomal Sequestration of Aminecontaining Drugs: Analysis and Therapeutic Implications. J Pharm Sci 2007:96:729-46.
28. He S, Li Q, Jiang X, Lu X, Feng F, Qu W, et al. Design of Small Molecule Autophagy Modulators: A Promising Druggable Strategy. J Med Chem 2018:61:4656-87.
29. Selvam T P, Kumar P V. Quinazoline Marketed drugs-A Review Res Pharm 2011:1:1-21.
30. Zhou D, Dai S-M, Tong Q. COVID-19: a Recommendation to Examine the Effect of Hydroxychloroquine in Preventing Infection and Progression. J Antimicrob Chemother 2020: doi: 10.1093/jac/dkaal 14.
31. Golden E B, Cho H Y, Hofman F M, Louie S G, Schonthal A H, Chen T C. Quinoline-based Antimalarial Drugs: A Novel Class of Autophagy Inhibitors. Neurosurg Focus 2015:38:E12.
32. Wang T, Goodall M L, Gonzales P, Sepulveda M, Martin K R, Gately S, et al. Synthesis of Improved Lysomotropic Autophagy Inhibitors. J Med Chem 2015:58:3025-35.
33. Chamberlain B T, Rice J M, Jernigan F E, III, Sherman W, Kulkarni M M, Shechter S, et al.: Oxoacridinylacetic Acid Derivatives as STING Protein Modulators and Their Preparation. USA2008.
34. Wang P, Rao H, Hua R, Li C-J. Rhodium-Catalyzed Xanthone Formation from 2-Aryloxy benzaldehydes via Cross-Dehydrogenative Coupling (CDC). Org Lett 2012:14:902-5.
35. Lubberstedt M, Müller-Vieira U, Mayer M, Biemel K M, Knospel F, Knobeloch D, et al. HepaRG human hepatic cell line utility as a surrogate for primary human hepatocytes in drug metabolism assessment in vitro. J Pharmacol Toxicol Methods 2011: 63 (1): 59-68 doi 10.1016/j.vascn.2010.04.013.
36. Kathawala R J, Espitia C M, Jones T M, Islam S, Gupta P, Zhang Y K, et al. ABCG2 Overexpression Contributes to Pevonedistat Resistance. Cancers (Basel) 2020: 12 (2) doi 10.3390/cancers 12020429.
37. Kishimoto W, Ishiguro N, Ludwig-Schwellinger E, Ebner T, Maeda K, Sugiyama Y. Usefulness of A Model-Based Approach for Estimating In Vitro P-Glycoprotein Inhibition Potency in a Transcellular Transport Assay. J Pharm Sci 2016: 105 (2): 891-6 doi 10.1016/j.xphs.2015.11.006.
38. Schmitt M V, Lienau P, Fricker G, Reichel A. Quantitation of Lysosomal Trapping of Basic Lipophilic Compounds Using In Vitro Assays and In Silico Predictions Based on the Determination of the Full Ph Profile of the Endo-/Lysosomal System in Rat Hepatocytes. Drug Metab Dispos 2019;47 (1): 49-57 doi 10.1124/dmd.118.084541.
39. Nawrocki S T, Kelly K R, Smith P G, Keaton M, Carraway H, Sekeres M A, et al. The NEDD8-activating enzyme inhibitor MLN4924 disrupts nucleotide metabolism and augments the efficacy of cytarabine. Clin Cancer Res 2015;21 (2): 439-47 doi 10.1158/1078-0432.CCR-14-1960.
40. Swords R T, Kelly K R, Smith P G, Garnsey J J, Mahalingam D, Medina E, et al. Inhibition of NEDD8-activating enzyme: a novel approach for the treatment of acute myeloid leukemia. Blood 2010; 115 (18): 3796-800 doi blood-2009-11-254862 [pii]10.1182/blood-2009-11-254862.
41. Smith E, Collins I. Photoaffinity labeling in target- and binding-site identification. Future Med Chem 2015;7 (2): 159-83 doi 10.4155/fmc. 14.152.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within Formula I: 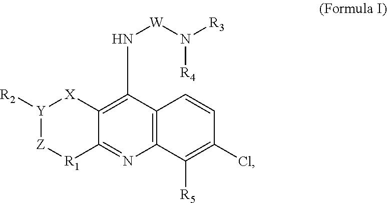
including pharmaceutically acceptable salts, solvates, and/or prodrugs thereof,
wherein each of W, X, Y, Z, R₁, R₂, R₃, R₄, and R₅ independently include any chemical moiety that permits the resulting compound to inhibit cellular autophagy and/or HDAC activity.

2. The compound of claim 1, wherein W is selected from

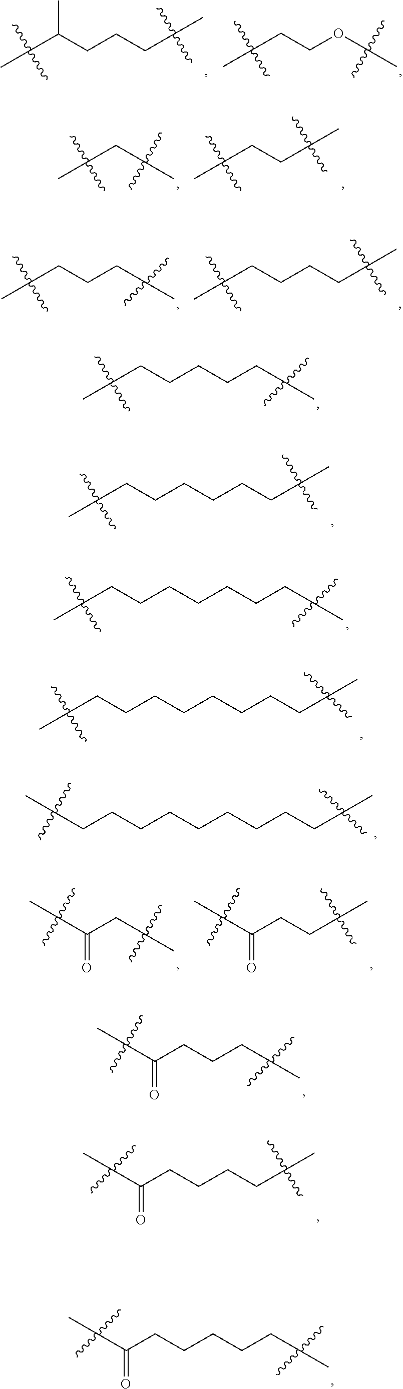

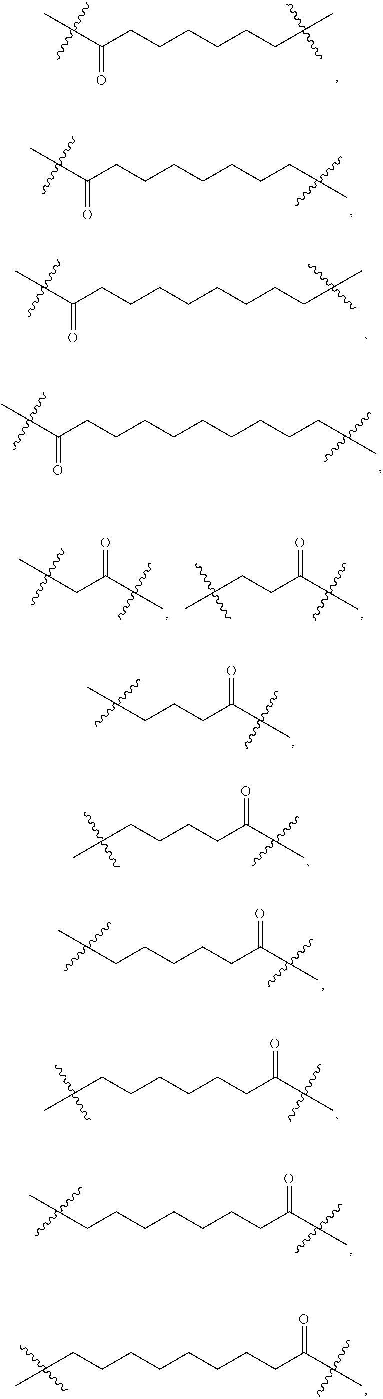

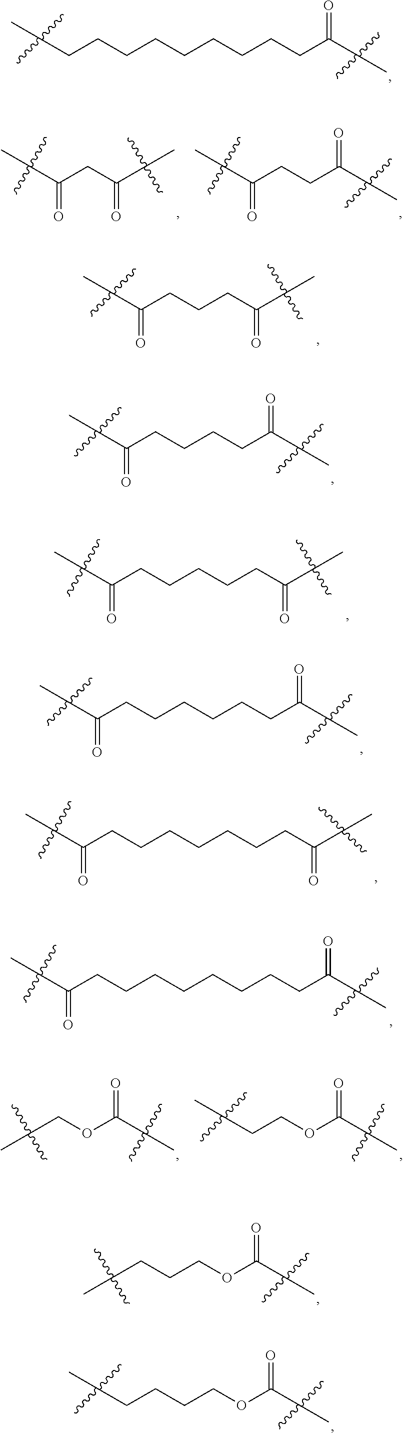

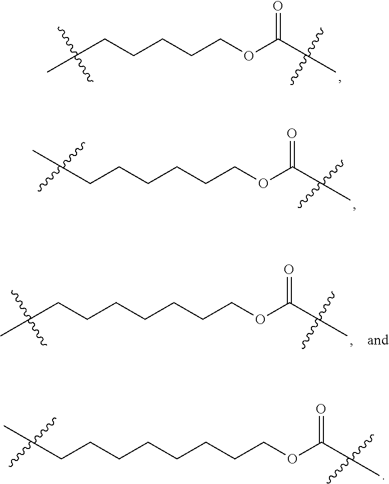 [SMILES: *CCCCCOC(*)=O ; *CCCCCCOC(*)=O ; *CCCCCCCOC(*)=O ; *CCCCCCCCOC(*)=O]

3. The compound of claim 1,
wherein X, Y, and Z are CH such that the resulting compound is represented by Formula II: 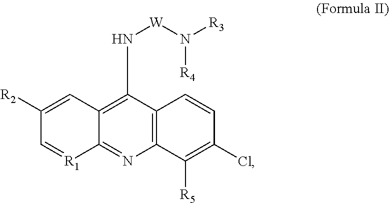
or
wherein X and Z are CH₂, and Y is CH such that the resulting compound is represented by Formula III:

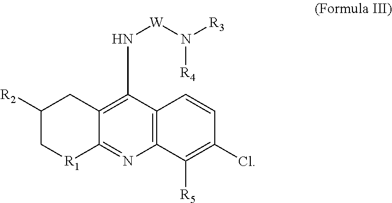

4. The compound of claim 1, wherein R1 is CH₂, N, N-alkyl, NCO, or NH.

5. The compound of claim 1, wherein R2 is methoxy, halogen, NH2, alkyl, aryl, or hydrogen.

6. The compound of claim 1, wherein R3 is selected from hydrogen, methyl, ethyl,

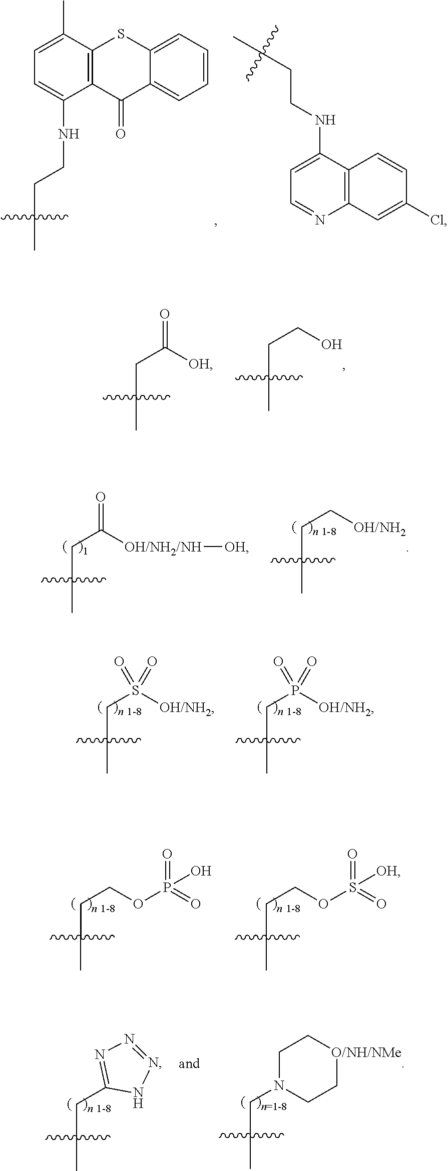

7. The compound of claim 1, wherein R4 is selected from hydrogen, methyl, ethyl, hydroxyl,

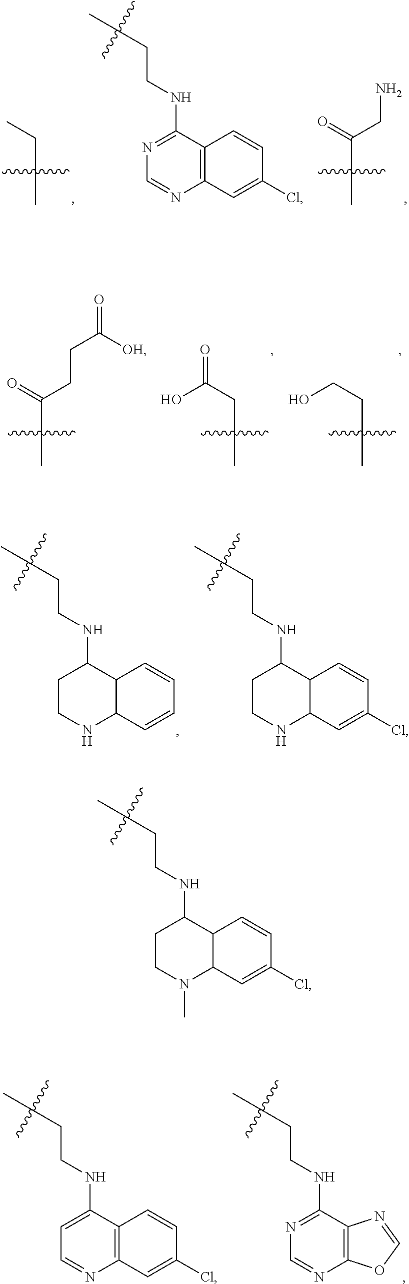

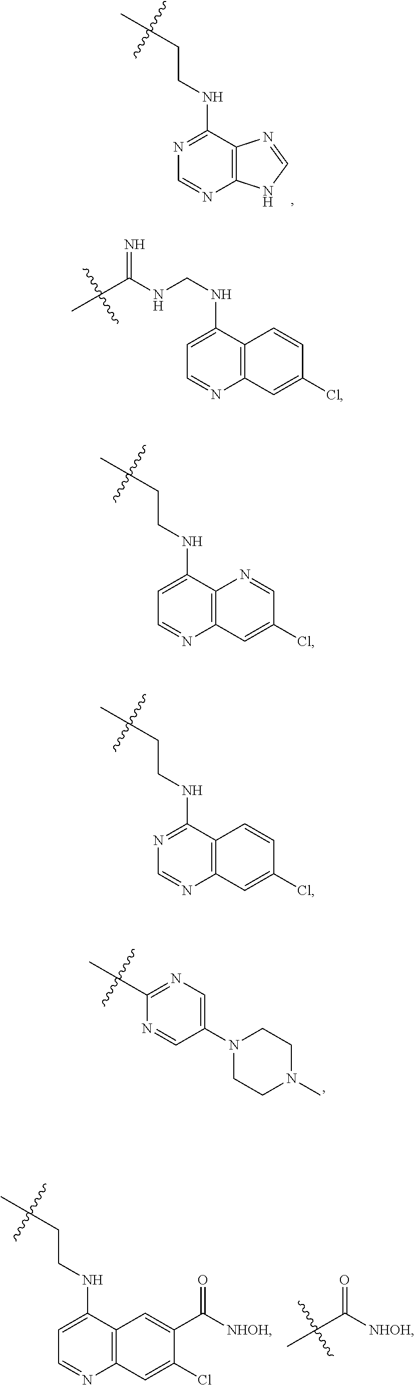

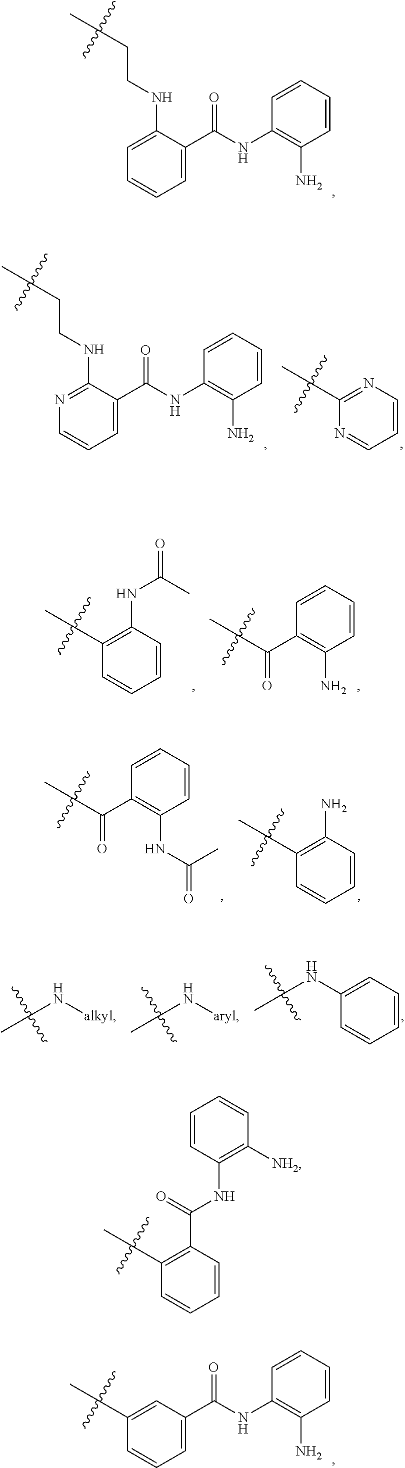

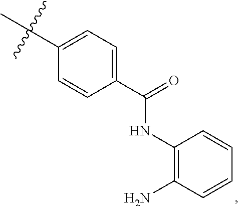 [SMILES: *c1ccc(C(=O)Nc2ccccc2N)cc1]

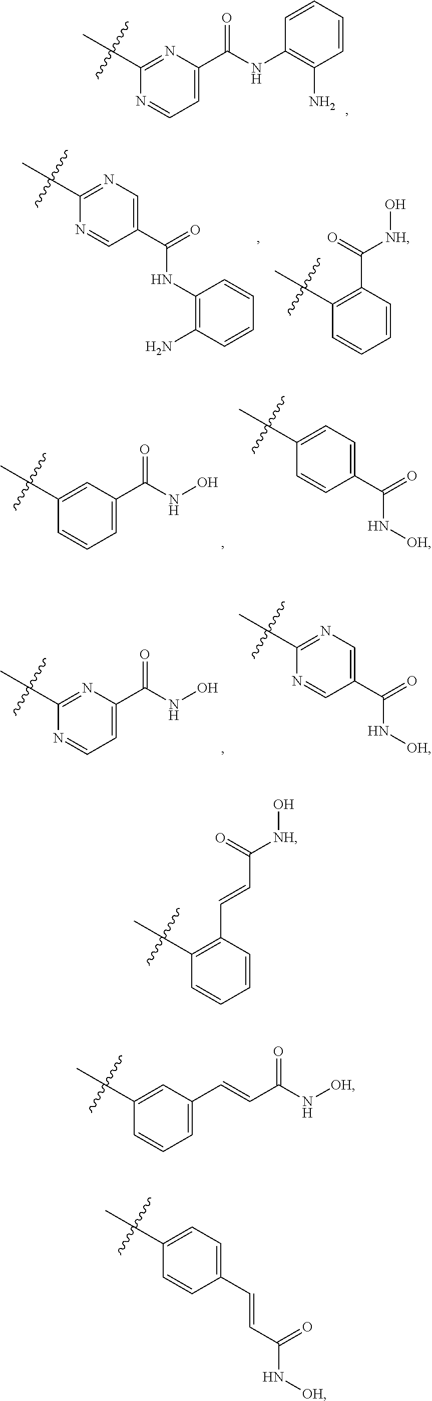

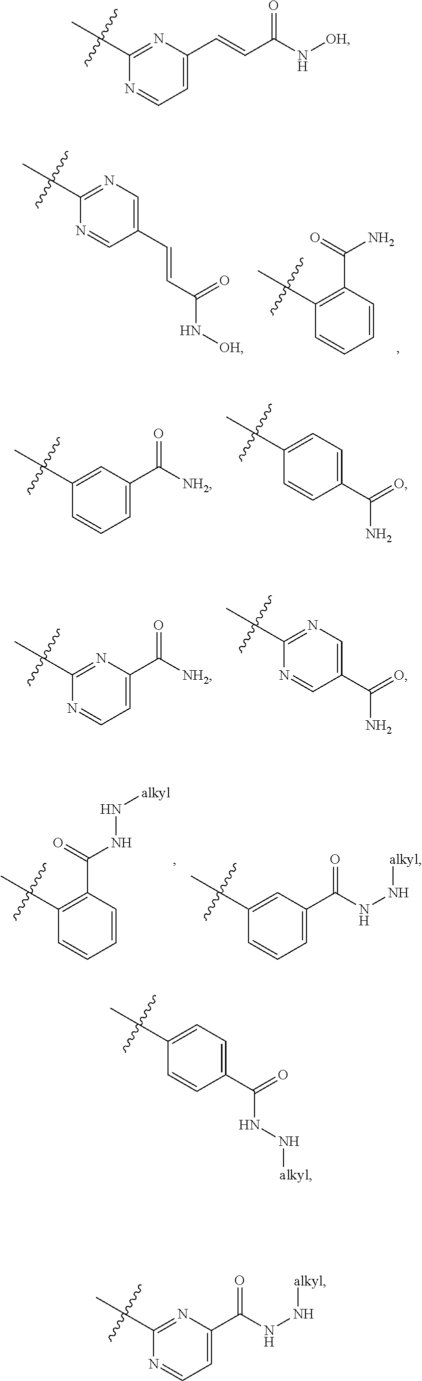

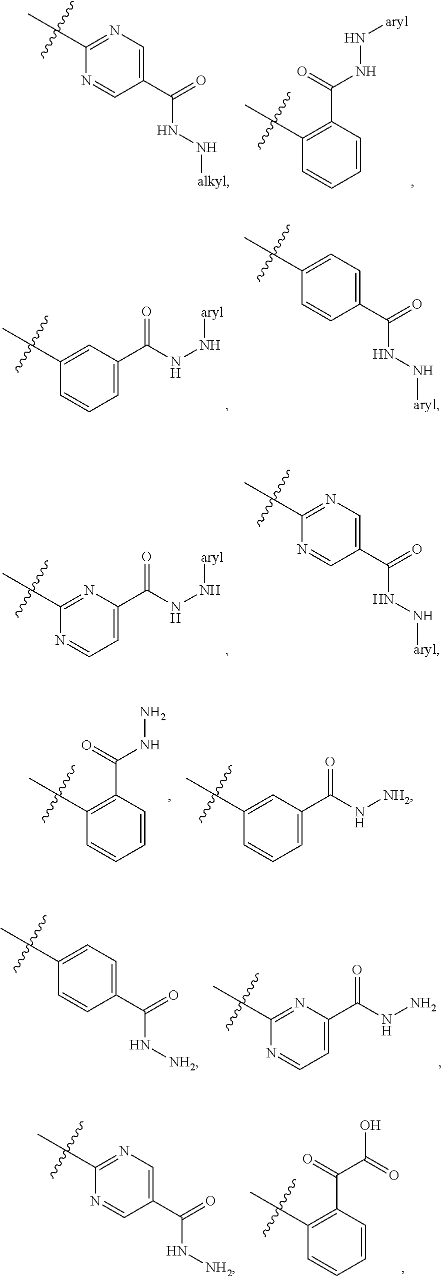

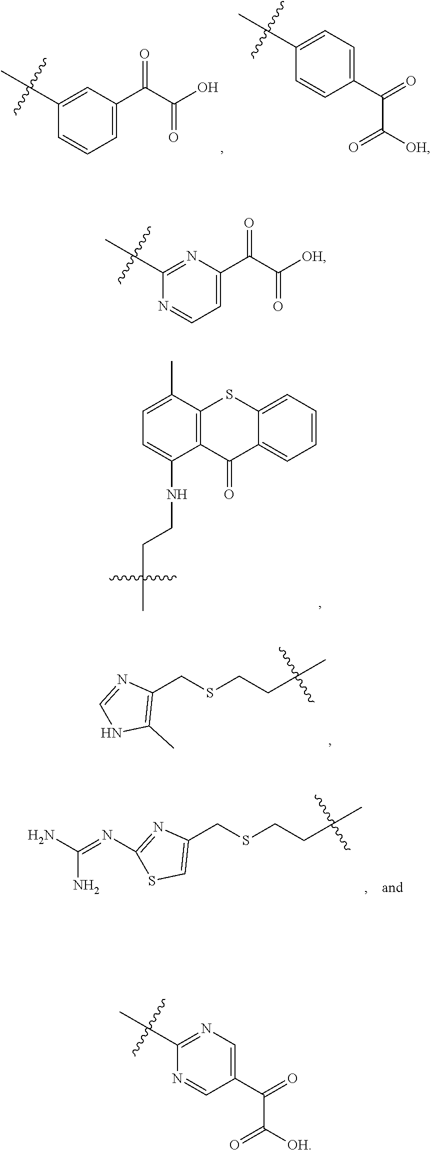

8. The compound of claim 1, wherein R5 is selected from hydrogen, methyl, and deuterated methyl (e.g., CD₃).

9. The compound of claim 1, wherein each of W, X, Y, Z, R₁, R₂, R₃, R₄, and R₅ independently include any chemical moiety that permits the resulting compound to act as an effective therapeutic for treating a condition characterized with aberrant autophagy activity and/or aberrant HDAC activity.

10. The compound of claim 9, wherein the condition characterized with aberrant autophagy activity and/or aberrant HDAC activity is selected from cancer, pulmonary hypertension, diabetes, neurodegenerative disorders, aging, heart disease, rheumatoid arthritis, infectious diseases, conditions and symptoms caused by a viral infection (e.g., COVID-19).

11. A pharmaceutical composition comprising a compound of claim 1.

12. A method of treating, ameliorating, or preventing a disorder related to aberrant cellular autophagy and/or HDAC activity in a patient comprising administering to said patient a therapeutically effective amount of the pharmaceutical composition of claim 11.

13. The method of claim 12, wherein said disorder is selected from cancer, pulmonary hypertension, diabetes, neurodegenerative disorders, aging, heart disease, rheumatoid arthritis, and infectious diseases.

14. The method of claim 12, wherein said patient is a human patient.

15. The method of claim 12, further comprising administering to said patient one or more agents for treating the disorder.

16. A kit comprising a compound of claim 1 and instructions for administering said compound to a patient having a disorder related to aberrant cellular autophagy and/or HDAC activity.

17. A method for inhibiting cellular autophagy and/or HDAC activity in a subject, comprising administering to the subject a compound of claim 1.

18. A method for treating, ameliorating and/or preventing a condition related to viral infection in a subject, comprising administering to the subject a pharmaceutical composition of claim 11.

19. The method of claim 18, wherein the condition related to viral infection is SARS-CoV-2 infection (e.g., COVID-19).

20. The method of claim 18, wherein the subject is a human subject suffering from or at risk of suffering from a condition related to SARS-COV-2 infection (e.g., COVID-19).

21. The method of claim 18, wherein the pharmaceutical composition is dispersed in a pharmaceutically acceptable carrier.

22. The method of claim 18, wherein the administering is oral, topical or intravenous.

23. The method of claim 18, further comprising administering to the subject remdesivir and/or hydroxychloroquine.

24. A method for treating, ameliorating and/or preventing symptoms related to viral infection in a subject, comprising administering to the subject a pharmaceutical composition of claim 11.

25. The method of claim 24, wherein the symptoms related to viral infection in a subject are one or more of fever, fatigue, dry cough, myalgias, dyspnea, acute respiratory distress syndrome, and pneumonia.

26. The method of claim 24, wherein the subject is a human subject suffering from or at risk of suffering from a condition related to SARS-COV-2 infection (e.g., COVID-19).

27. The method of claim 24, wherein the pharmaceutical composition is dispersed in a pharmaceutically acceptable carrier.

28. The method of claim 24, wherein the administering is oral, intravenous, or topical.

29. The method of claim 24, further comprising administering to the subject remdesivir and/or hydroxychloroquine.

30. A method for treating, ameliorating and/or preventing acute respiratory distress syndrome in a subject, comprising administering to the subject a pharmaceutical composition of claim 11.

31. The method of claim 30, wherein the acute respiratory distress syndrome is related to SARS-COV-2 infection (e.g., COVID-19).

32. The method of claim 30, wherein the subject is a human subject suffering from or at risk of suffering from a condition related to SARS-COV-2 infection (e.g., COVID-19).

33. The method of claim 30, wherein the administering is oral, intravenous or topical.

34. The method of claim 30, further comprising administering to the subject remdesivir and/or hydroxychloroquine.

35. A method for treating, ameliorating and/or preventing pneumonia in a subject, comprising administering to the subject a pharmaceutical composition of claim 11.

36. The method of claim 35, wherein the pneumonia is related to SARS-COV-2 infection (e.g., COVID-19).

37. The method of claim 35, wherein the subject is a human subject suffering from or at risk of suffering from a condition related to SARS-COV-2 infection (e.g., COVID-19).

38. The method of claim 35, wherein the administering is oral, intravenous or topical.

39. The method of claim 35, further comprising administering an additional agent for treating pneumonia.

40. The method of claim 35, further comprising administering to the subject remdesivir and/or hydroxychloroquine.

41. A kit comprising (1) a pharmaceutical composition of claim 11, (2) a container, pack, or dispenser, and (3) instructions for administration.

42. The kit of claim 41, wherein kit further comprises remdesivir and/or hydroxychloroquine.
